(19) United States
(12) Patent Application Publication
(10) Pub. No.: US 2024/0194340 A1
(43) Pub. Date: Jun. 13, 2024

(54) METHODS AND SYSTEMS FOR CUSTOMIZING TREATMENTS

(71) Applicant: KPN INNOVATIONS, LLC., LAKEWOOD, CO (US)

(72) Inventor: Kenneth Neumann, LAKEWOOD, CO (US)

(73) Assignee: KPN INNOVATIONS, LLC., LAKEWOOD, CO (US)

(21) Appl. No.: 18/410,381

(22) Filed: Jan. 11, 2024

Related U.S. Application Data
(63) Continuation-in-part of application No. 17/977,190, filed on Oct. 31, 2022, now Pat. No. 11,887,735.
Application No. 17/977,190 is a continuation of application No. 16/727,099, filed on Dec. 26, 2019, now Pat. No. 11,562,828.

(51) Int. Cl. G16H 50/20

(52) U.S. Cl. CPC G16H 50/20

(57) ABSTRACT

A system for customizing treatments. The system includes a computing device configured to record a user biological extraction containing an element of user physiological data. The computing device is configured to receive condition state training data and generate a condition state model utilizing a first machine-learning algorithm. The computing device is configured to calculate a condition state label using the condition state model. The computing device is configured to select a treatment model utilizing the condition state label. The computing device is configured to generate a treatment model and output a plurality of treatments utilizing the treatment model.

### CROSS-REFERENCE TO RELATED APPLICATIONS

This application is a continuation-in-part of Non-provisional application Ser. No. 17/977,190, filed on Oct. 31, 2022, and entitled “METHODS AND SYSTEMS FOR CUSTOMIZING TREATMENTS,” which is a continuation of Non-provisional application Ser. No. 16/727,099, filed on Dec. 26, 2019, and entitled “METHODS AND SYSTEMS FOR CUSTOMIZING TREATMENTS,” the entirety of which is incorporated herein by reference.

### FIELD OF THE INVENTION

The present invention generally relates to the field of artificial intelligence. In particular, the present invention is directed to methods and systems for customizing treatments.

### BACKGROUND

Frequently, treatments are arbitrarily recommended based on current trends and stale literature. On occasion, uninformed treatment selection and implementation leads to adverse effects that create further harm as opposed to solving an underlying problem. Currently, there lacks measures that can detect and recommend treatments that are customized and unique to each individual.

### SUMMARY OF THE DISCLOSURE

In an aspect a system for customizing treatments, the system including a computing device, the computing device designed and configured to calculate a condition state label as a function of an element of user physiological data. The computing device further configured to generate a treatment model, using a second machine-learning algorithm and a treatment training set, wherein the treatment model utilizes condition state labels as inputs and outputs treatments, wherein generating the treatment model further includes calculating a treatment category selector as a function of an implementation factor, wherein the implementation factor indicates a user preference pertaining to different treatment practices. The computing device further configured to output a treatment utilizing the treatment model. The computing device further configured to receive a treatment response from a remote device. The computing device further configured to generate a treatment response score as a function of the treatment response.

In an aspect, a method for customizing treatments, the method including calculating, by the computing device, a condition state label as a function of an element of user physiological data. The method further including generating, by the computing device, a treatment model, using a second machine-learning algorithm and a treatment training set, wherein the treatment model utilizes condition state labels as inputs and outputs treatments, wherein generating the treatment model further includes calculating a treatment category selector as a function of an implementation factor, wherein the implementation factor indicates a user preference pertaining to different treatment practices. The method further including outputting, by the computing device, a treatment utilizing the treatment model. The method further including receiving, by the computing device, a treatment response from a remote device. The method further including generating, by the computing device, a treatment response score as a function of the treatment response.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for customizing treatments;

FIG. 2 is a block diagram illustrating an exemplary embodiment of a user database;

FIG. 3 is a block diagram illustrating an exemplary embodiment of a machine-learning database;

FIG. 4 is a block diagram illustrating an exemplary embodiment of an expert knowledge database;

FIG. 5 is a block diagram illustrating an exemplary embodiment of a machine-learning module;

FIG. 6 is a diagram of an exemplary embodiment of a neural network;

FIG. 7 is a diagram of an exemplary embodiment of a node of a neural network;

FIG. 8 is a process flow diagram illustrating an exemplary embodiment of a method of customizing treatments; and

FIG. 9 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, embodiments disclosed herein utilize a human subject's biological extraction to propose suggested treatments. Suggested treatments are determined to be compatible and uniquely suggested for each human subject.

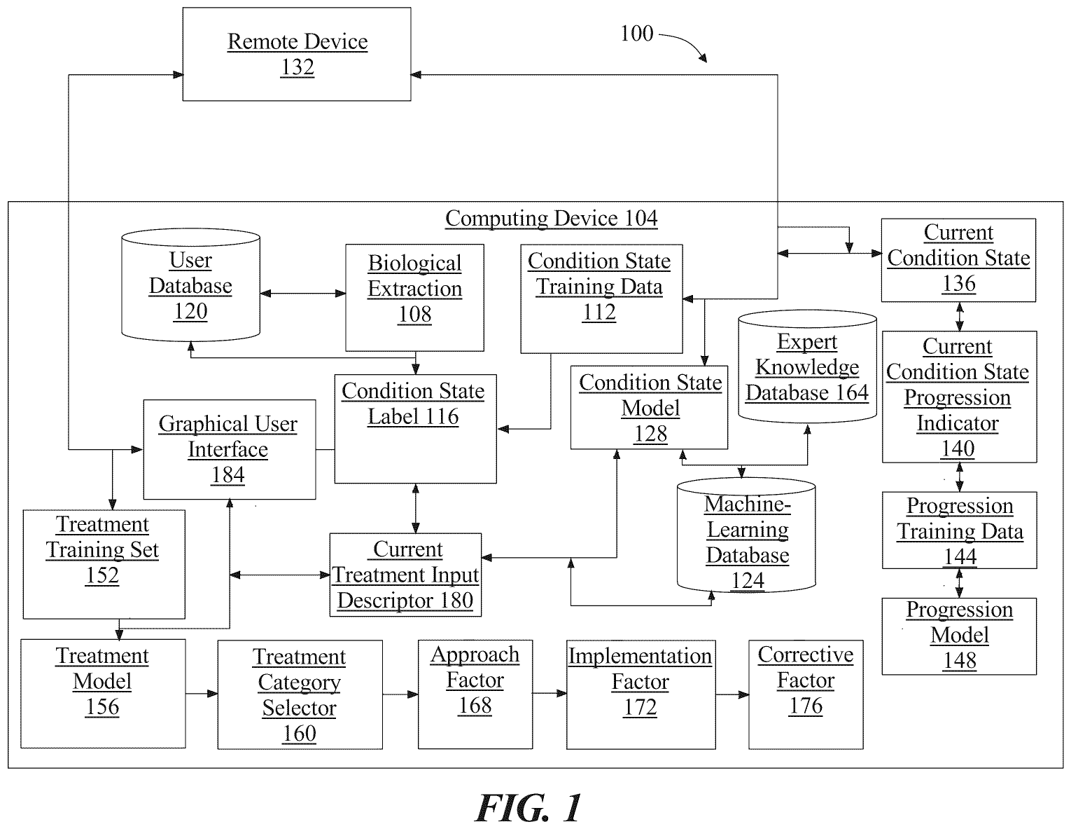

Referring now to FIG. 1, an exemplary embodiment of a system 100 for customizing treatments is illustrated. System 100 includes a computing device 104. Computing device 104 may include any computing device 104 as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing device 104 operating independently or may include two or more computing device 104 operating in concert, in parallel, sequentially or the like; two or more computing devices 104 may be included together in a single computing device 104 or in two or more computing devices 104. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices 104, and any combinations thereof. A network may employ a wired and/or a wireless mode of communication. In general, any network topology may be used. Information (e.g., data, software etc.) may be communicated to and/or from a computer and/or a computing device 104. Computing device 104 may include but is not limited to, for example, a computing device 104 or cluster of computing devices 104 in a first location and a second computing device 104 or cluster of computing devices 104 in a second location. Computing device 104 may include one or more computing devices 104 dedicated to data storage, security, distribution of traffic for load balancing, and the like. Computing device 104 may distribute one or more computing tasks as described below across a plurality of computing devices 104 of computing device 104, which may operate in parallel, in series, redundantly, or in any other manner used for distribution of tasks or memory between computing devices 104. Computing device 104 may be implemented using a “shared nothing” architecture in which data is cached at the worker; in an embodiment, this may enable scalability of system 100 and/or computing device 104.

Still referring to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, computing device 104 is configured to record a user biological extraction 108 containing at least an element of user physiological data. A “biological extraction” as used in this disclosure includes at least an element of user physiological data. As used in this disclosure, “physiological data” is any data indicative of a person's physiological state; physiological state may be evaluated with regard to one or more measures of health of a person's body, one or more systems within a person's body such as a circulatory system, a digestive system, a nervous system, or the like, one or more organs within a person's body, and/or any other subdivision of a person's body useful for diagnostic or prognostic purposes. A user biological extraction may contain a plurality of elements of user physiological data, such as for example several biomarkers obtained from a blood test or multiple biomarkers obtained from a saliva sample. For instance, and without limitation, a particular set of biomarkers, test results, and/or biochemical information may be recognized in a given medical field as useful for identifying various disease conditions or prognoses within a relevant field. As a non-limiting example, and without limitation, physiological data describing red blood cells, such as red blood cell count, hemoglobin levels, hematocrit, mean corpuscular volume, mean corpuscular hemoglobin, and/or mean corpuscular hemoglobin concentration may be recognized as useful for identifying various conditions such as dehydration, high testosterone, nutrient deficiencies, kidney dysfunction, chronic inflammation, anemia, and/or blood loss.

With continued reference to FIG. 1, physiological state data may include, without limitation, hematological data, such as red blood cell count, which may include a total number of red blood cells in a person's blood and/or in a blood sample, hemoglobin levels, hematocrit representing a percentage of blood in a person and/or sample that is composed of red blood cells, mean corpuscular volume, which may be an estimate of the average red blood cell size, mean corpuscular hemoglobin, which may measure average weight of hemoglobin per red blood cell, mean corpuscular hemoglobin concentration, which may measure an average concentration of hemoglobin in red blood cells, platelet count, mean platelet volume which may measure the average size of platelets, red blood cell distribution width, which measures variation in red blood cell size, absolute neutrophils, which measures the number of neutrophil white blood cells, absolute quantities of lymphocytes such as B-cells, T-cells, Natural Killer Cells, and the like, absolute numbers of monocytes including macrophage precursors, absolute numbers of eosinophils, and/or absolute counts of basophils. Physiological state data may include, without limitation, immune function data such as Interleukine-6 (IL-6), TNF-alpha, systemic inflammatory cytokines, and the like.

Continuing to refer to FIG. 1, physiological state data may include, without limitation, data describing blood-born lipids, including total cholesterol levels, high-density lipoprotein (HDL) cholesterol levels, low-density lipoprotein (LDL) cholesterol levels, very low-density lipoprotein (VLDL) cholesterol levels, levels of triglycerides, and/or any other quantity of any blood-born lipid or lipid-containing substance. Physiological state data may include measures of glucose metabolism such as fasting glucose levels and/or hemoglobin A1-C(HbA1c) levels. Physiological state data may include, without limitation, one or more measures associated with endocrine function, such as without limitation, quantities of dehydroepiandrosterone (DHEAS), DHEA-Sulfate, quantities of cortisol, ratio of DHEAS to cortisol, quantities of testosterone quantities of estrogen, quantities of growth hormone (GH), insulin-like growth factor 1 (IGF-1), quantities of adipokines such as adiponectin, leptin, and/or ghrelin, quantities of somatostatin, progesterone, or the like. Physiological state data may include measures of estimated glomerular filtration rate (eGFR). Physiological state data may include quantities of C-reactive protein, estradiol, ferritin, folate, homocysteine, prostate-specific Ag, thyroid-stimulating hormone, vitamin D, 25 hydroxy, blood urea nitrogen, creatinine, sodium, potassium, chloride, carbon dioxide, uric acid, albumin, globulin, calcium, phosphorus, alkaline phosphatase, alanine amino transferase, aspartate amino transferase, lactate dehydrogenase (LDH), bilirubin, gamma-glutamyl transferase (GGT), iron, and/or total iron binding capacity (TIBC), or the like. Physiological state data may include antinuclear antibody levels. Physiological state data may include aluminum levels. Physiological state data may include arsenic levels. Physiological state data may include levels of fibrinogen, plasma cystatin C, and/or brain natriuretic peptide.

Continuing to refer to FIG. 1, physiological state data may include measures of lung function such as forced expiratory volume, one second (FEV-1) which measures how much air can be exhaled in one second following a deep inhalation, forced vital capacity (FVC), which measures the volume of air that may be contained in the lungs. Physiological state data may include a measurement blood pressure, including without limitation systolic and diastolic blood pressure. Physiological state data may include a measure of waist circumference. Physiological state data may include body mass index (BMI). Physiological state data may include one or more measures of bone mass and/or density such as dual-energy x-ray absorptiometry. Physiological state data may include one or more measures of muscle mass. Physiological state data may include one or more measures of physical capability such as without limitation measures of grip strength, evaluations of standing balance, evaluations of gait speed, pegboard tests, timed up and go tests, and/or chair rising tests.

Still viewing FIG. 1, physiological state data may include one or more measures of cognitive function, including without limitation Rey auditory verbal learning test results, California verbal learning test results, NIH toolbox picture sequence memory test, Digital symbol coding evaluations, and/or Verbal fluency evaluations. Physiological state data may include one or more evaluations of sensory ability, including measures of audition, vision, olfaction, gustation, vestibular function and pain.

Continuing to refer to FIG. 1, physiological state data may include psychological data. Psychological data may include any data generated using psychological, neuro-psychological, and/or cognitive evaluations, as well as diagnostic screening tests, personality tests, personal compatibility tests, or the like; such data may include, without limitation, numerical score data entered by an evaluating professional and/or by a subject performing a self-test such as a computerized questionnaire. Psychological data may include textual, video, or image data describing testing, analysis, and/or conclusions entered by a medical professional such as without limitation a psychologist, psychiatrist, psychotherapist, social worker, a medical doctor, or the like. Psychological data may include data gathered from user interactions with persons, documents, and/or computing devices; for instance, user patterns of purchases, including electronic purchases, communication such as via chat-rooms or the like, any textual, image, video, and/or data produced by the subject, any textual image, video and/or other data depicting and/or describing the subject, or the like. Any psychological data and/or data used to generate psychological data may be analyzed using machine-learning and/or language processing module as described in this disclosure.

Still referring to FIG. 1, physiological state data may include genomic data, including deoxyribonucleic acid (DNA) samples and/or sequences, such as without limitation DNA sequences contained in one or more chromosomes in human cells. Genomic data may include, without limitation, ribonucleic acid (RNA) samples and/or sequences, such as samples and/or sequences of messenger RNA (mRNA) or the like taken from human cells. Genetic data may include telomere lengths. Genomic data may include epigenetic data including data describing one or more states of methylation of genetic material. Physiological state data may include proteomic data, which, as used herein, is data describing all proteins produced and/or modified by an organism, colony of organisms, or system of organisms, and/or a subset thereof. Physiological state data may include data concerning a microbiome of a person, which as used herein includes any data describing any microorganism and/or combination of microorganisms living on or within a person, including without limitation biomarkers, genomic data, proteomic data, and/or any other metabolic or biochemical data useful for analysis of the effect of such microorganisms on other physiological state data of a person, as described in further detail below.

With continuing reference to FIG. 1, physiological state data may include one or more user-entered descriptions of a person's physiological state. One or more user-entered descriptions may include, without limitation, user descriptions of symptoms, which may include without limitation current or past physical, psychological, perceptual, and/or neurological symptoms, user descriptions of current or past physical, emotional, and/or psychological problems and/or concerns, user descriptions of past or current treatments, including therapies, nutritional regimens, exercise regimens, pharmaceuticals or the like, or any other user-entered data that a user may provide to a medical professional when seeking treatment and/or evaluation, and/or in response to medical intake papers, questionnaires, questions from medical professionals, or the like. Physiological state data may include any physiological state data, as described above, describing any multicellular organism living in or on a person including any parasitic and/or symbiotic organisms living in or on the persons; non-limiting examples may include mites, nematodes, flatworms, or the like. Examples of physiological state data described in this disclosure are presented for illustrative purposes only and are not meant to be exhaustive.

With continued reference to FIG. 1, physiological data may include, without limitation any result of any medical test, physiological assessment, cognitive assessment, psychological assessment, or the like. System 100 may receive at least a physiological data from one or more other devices after performance; system 100 may alternatively or additionally perform one or more assessments and/or tests to obtain at least a physiological data, and/or one or more portions thereof, on system 100. For instance, at least physiological data may include or more entries by a user in a form or similar graphical user interface 184 object; one or more entries may include, without limitation, user responses to questions on a psychological, behavioral, personality, or cognitive test. For instance, at least a server 104 may present to user a set of assessment questions designed or intended to evaluate a current state of mind of the user, a current psychological state of the user, a personality trait of the user, or the like; at least a server 104 may provide user-entered responses to such questions directly as at least a physiological data and/or may perform one or more calculations or other algorithms to derive a score or other result of an assessment as specified by one or more testing protocols, such as automated calculation of a Stanford-Binet and/or Wechsler scale for IQ testing, a personality test scoring such as a Myers-Briggs test protocol, or other assessments that may occur to persons skilled in the art upon reviewing the entirety of this disclosure.

With continued reference to FIG. 1, assessment and/or self-assessment data, and/or automated or other assessment results, obtained from a third-party device; third-party device may include, without limitation, a server or other device (not shown) that performs automated cognitive, psychological, behavioral, personality, or other assessments. Third-party device may include a device operated by an informed advisor. An informed advisor may include any medical professional who may assist and/or participate in the medical treatment of a user. An informed advisor may include a medical doctor, nurse, physician assistant, pharmacist, yoga instructor, nutritionist, spiritual healer, meditation teacher, fitness coach, health coach, life coach, and the like.

With continued reference to FIG. 1, physiological data may include data describing one or more test results, including results of mobility tests, stress tests, dexterity tests, endocrinal tests, genetic tests, and/or electromyographic tests, biopsies, radiological tests, genetic tests, and/or sensory tests. Persons skilled in the art, upon reviewing the entirety of this disclosure, will be aware of various additional examples of at least a physiological sample consistent with this disclosure.

With continued reference to FIG. 1, physiological data may include one or more user body measurements. A “user body measurement” as used in this disclosure, includes a measurable indicator of the severity, absence, and/or presence of a disease state. A “disease state” as used in this disclosure, includes any harmful deviation from the normal structural and/or function state of a human being. A disease state may include any medical condition and may be associated with specific symptoms and signs. A disease state may be classified into different types including infectious diseases, deficiency diseases, hereditary diseases, and/or physiological diseases. For instance and without limitation, internal dysfunction of the immune system may produce a variety of different diseases including immunodeficiency, hypersensitivity, allergies, and/or autoimmune disorders.

With continued reference to FIG. 1, user body measurements may be related to particular dimensions of the human body. A “dimension of the human body” as used in this disclosure, includes one or more functional body systems that are impaired by disease in a human body and/or animal body. Functional body systems may include one or more body systems recognized as attributing to root causes of disease by functional medicine practitioners and experts. A “root cause” as used in this disclosure, includes any chain of causation describing underlying reasons for a particular disease state and/or medical condition instead of focusing solely on symptomatology reversal. Root cause may include chains of causation developed by functional medicine practices that may focus on disease causation and reversal. For instance and without limitation, a medical condition such as diabetes may include a chain of causation that does not include solely impaired sugar metabolism but that also includes impaired hormone systems including insulin resistance, high cortisol, less than optimal thyroid production, and low sex hormones. Diabetes may include further chains of causation that include inflammation, poor diet, delayed food allergies, leaky gut, oxidative stress, damage to cell membranes, and dysbiosis. Dimensions of the human body may include but are not limited to epigenetics, gut-wall, microbiome, nutrients, genetics, and/or metabolism.

With continued reference to FIG. 1, epigenetic, as used herein, includes any user body measurements describing changes to a genome that do not involve corresponding changes in nucleotide sequence. Epigenetic body measurement may include data describing any heritable phenotypic. Phenotype, as used herein, include any observable trait of a user including morphology, physical form, and structure. Phenotype may include a user's biochemical and physiological properties, behavior, and products of behavior. Behavioral phenotypes may include cognitive, personality, and behavior patterns. This may include effects on cellular and physiological phenotypic traits that may occur due to external or environmental factors. For example, DNA methylation and histone modification may alter phenotypic expression of genes without altering underlying DNA sequence. Epigenetic body measurements may include data describing one or more states of methylation of genetic material.

With continued reference to FIG. 1, gut-wall, as used herein, includes the space surrounding the lumen of the gastrointestinal tract that is composed of four layers including the mucosa, submucosa, muscular layer, and serosa. The mucosa contains the gut epithelium that is composed of goblet cells that function to secrete mucus, which aids in lubricating the passage of food throughout the digestive tract. The goblet cells also aid in protecting the intestinal wall from destruction by digestive enzymes. The mucosa includes villi or folds of the mucosa located in the small intestine that increase the surface area of the intestine. The villi contain a lacteal, that is a vessel connected to the lymph system that aids in removal of lipids and tissue fluids. Villi may contain microvilli that increase the surface area over which absorption can take place. The large intestine lack villi and instead a flat surface containing goblet cells are present.

With continued reference to FIG. 1, gut-wall includes the submucosa, which contains nerves, blood vessels, and elastic fibers containing collagen. Elastic fibers contained within the submucosa aid in stretching the gastrointestinal tract with increased capacity while also maintaining the shape of the intestine. Gut-wall includes muscular layer which contains smooth muscle that aids in peristalsis and the movement of digested material out of and along the gut. Gut-wall includes the serosa which is composed of connective tissue and coated in mucus to prevent friction damage from the intestine rubbing against other tissue. Mesenteries are also found in the serosa and suspend the intestine in the abdominal cavity to stop it from being disturbed when a person is physically active.

With continued reference to FIG. 1, gut-wall body measurement may include data describing one or more test results including results of gut-wall function, gut-wall integrity, gut-wall strength, gut-wall absorption, gut-wall permeability, intestinal absorption, gut-wall barrier function, gut-wall absorption of bacteria, gut-wall malabsorption, gut-wall gastrointestinal imbalances and the like.

With continued reference to FIG. 1, gut-wall body measurement may include any data describing blood test results of creatinine levels, lactulose levels, zonulin levels, and mannitol levels. Gut-wall body measurement may include blood test results of specific gut-wall body measurements including d-lactate, endotoxin lipopolysaccharide (LPS) Gut-wall body measurement may include data breath tests measuring lactulose, hydrogen, methane, lactose, and the like. Gut-wall body measurement may include blood test results describing blood chemistry levels of albumin, bilirubin, complete blood count, electrolytes, minerals, sodium, potassium, calcium, glucose, blood clotting factors,

With continued reference to FIG. 1, gut-wall body measurement may include one or more stool test results describing presence or absence of parasites, firmicutes, Bacteroidetes, absorption, inflammation, food sensitivities. Stool test results may describe presence, absence, and/or measurement of acetate, aerobic bacterial cultures, anerobic bacterial cultures, fecal short chain fatty acids, beta-glucuronidase, cholesterol, chymotrypsin, fecal color, cryptosporidium EIA, Entamoeba histolytica, fecal lactoferrin, Giardia lamblia EIA, long chain fatty acids, meat fibers and vegetable fibers, mucus, occult blood, parasite identification, phospholipids, propionate, putrefactive short chain fatty acids, total fecal fat, triglycerides, yeast culture, n-butyrate, pH and the like.

With continued reference to FIG. 1, gut-wall body measurement may include one or more stool test results describing presence, absence, and/or measurement of microorganisms including bacteria, archaea, fungi, protozoa, algae, viruses, parasites, worms, and the like. Stool test results may contain species such as Bifidobacterium species, campylobacter species, Clostridium difficile, cryptosporidium species, Cyclospora cayetanensis, Cryptosporidium EIA, Dientamoeba fragilis, Entamoeba histolytica, Escherichia coli, Entamoeba histolytica, Giardia, H. pylori, Candida albicans, Lactobacillus species, worms, macroscopic worms, mycology, protozoa, Shiga toxin E. coli, and the like.

With continued reference to FIG. 1, gut-wall body measurement may include one or more microscopic ova exam results, microscopic parasite exam results, protozoan polymerase chain reaction test results and the like. Gut-wall body measurement may include enzyme-linked immunosorbent assay (ELISA) test results describing immunoglobulin G (Ig G) food antibody results, immunoglobulin E (Ig E) food antibody results, Ig E mold results, IgG spice and herb results. Gut-wall body measurement may include measurements of calprotectin, eosinophil protein x (EPX), stool weight, pancreatic elastase, total urine volume, blood creatinine levels, blood lactulose levels, blood mannitol levels.

With continued reference to FIG. 1, gut-wall body measurement may include one or more elements of data describing one or more procedures examining gut including for example colonoscopy, endoscopy, large and small molecule challenge and subsequent urinary recovery using large molecules such as lactulose, polyethylene glycol-3350, and small molecules such as mannitol, L-rhamnose, polyethyleneglycol-400. Gut-wall body measurement may include data describing one or more images such as x-ray, MRI, CT scan, ultrasound, standard barium follow-through examination, barium enema, barium with contract, MRI fluoroscopy, positron emission tomography 9PET), diffusion-weighted MRI imaging, and the like.

With continued reference to FIG. 1, microbiome, as used herein, includes ecological community of commensal, symbiotic, and pathogenic microorganisms that reside on or within any of a number of human tissues and biofluids. For example, human tissues and biofluids may include the skin, mammary glands, placenta, seminal fluid, uterus, vagina, ovarian follicles, lung, saliva, oral mucosa, conjunctiva, biliary, and gastrointestinal tracts. Microbiome may include for example, bacteria, archaea, protists, fungi, and viruses. Microbiome may include commensal organisms that exist within a human being without causing harm or disease. Microbiome may include organisms that are not harmful but rather harm the human when they produce toxic metabolites such as trimethylamine. Microbiome may include pathogenic organisms that cause host damage through virulence factors such as producing toxic by-products. Microbiome may include populations of microbes such as bacteria and yeasts that may inhabit the skin and mucosal surfaces in various parts of the body. Bacteria may include for example Firmicutes species, Bacteroidetes species, Proteobacteria species, Verrumicrobia species, Actinobacteria species, Fusobacteria species, Cyanobacteria species and the like. Archaea may include methanogens such as Methanobrevibacter smithies' and Methanosphaera stadtmanae. Fungi may include Candida species and Malassezia species. Viruses may include bacteriophages. Microbiome species may vary in different locations throughout the body. For example, the genitourinary system may contain a high prevalence of Lactobacillus species while the gastrointestinal tract may contain a high prevalence of Bifidobacterium species while the lung may contain a high prevalence of Streptococcus and Staphylococcus species.

With continued reference to FIG. 1, microbiome body measurement may include one or more stool test results describing presence, absence, and/or measurement of microorganisms including bacteria, archaea, fungi, protozoa, algae, viruses, parasites, worms, and the like. Stool test results may contain species such as Ackerman's muciniphila, Anaerotruncus colihominis, bacteriology, Bacteroides vulgates', Bacteroides-Prevotella, Barnesiella species, Bifidobacterium longarm, Bifidobacterium species, Butyrivbrio crossotus, Clostridium species, Collinsella aerofaciens, fecal color, fecal consistency, Coprococcus eutactus, Desulfovibrio piger, Escherichia coli, Faecalibacterium prausnitzii, Fecal occult blood, Firmicutes to Bacteroidetes ratio, Fusobacterium species, Lactobacillus species, Methanobrevibacter smithii, yeast minimum inhibitory concentration, bacteria minimum inhibitory concentration, yeast mycology, fungi mycology, Odoribacter species, Oxalobacter formigenes, parasitology, Prevotella species, Pseudoflavonifractor species, Roseburia species, Ruminococcus species, Veillonella species and the like.

With continued reference to FIG. 1, microbiome body measurement may include one or more stool tests results that identify all microorganisms living a user's gut including bacteria, viruses, archaea, yeast, fungi, parasites, and bacteriophages. Microbiome body measurement may include DNA and RNA sequences from live microorganisms that may impact a user's health. Microbiome body measurement may include high resolution of both species and strains of all microorganisms. Microbiome body measurement may include data describing current microbe activity. Microbiome body measurement may include expression of levels of active microbial gene functions. Microbiome body measurement may include descriptions of sources of disease causing microorganisms, such as viruses found in the gastrointestinal tract such as raspberry bushy swarf virus from consuming contaminated raspberries or Pepino mosaic virus from consuming contaminated tomatoes.

With continued reference to FIG. 1, microbiome body measurement may include one or more blood test results that identify metabolites produced by microorganisms. Metabolites may include for example, indole-3-propionic acid, indole-3-lactic acid, indole-3-acetic acid, tryptophan, serotonin, kynurenine, total indoxyl sulfate, tyrosine, xanthine, 3-methylxanthine, uric acid, and the like.

With continued reference to FIG. 1, microbiome body measurement may include one or more breath test results that identify certain strains of microorganisms that may be present in certain areas of a user's body. This may include for example, lactose intolerance breath tests, methane-based breath tests, hydrogen based breath tests, fructose based breath tests. Helicobacter pylori breath test, fructose intolerance breath test, bacterial overgrowth syndrome breath tests and the like.

With continued reference to FIG. 1, microbiome body measurement may include one or more urinary analysis results for certain microbial strains present in urine. This may include for example, urinalysis that examines urine specific gravity, urine cytology, urine sodium, urine culture, urinary calcium, urinary hematuria, urinary glucose levels, urinary acidity, urinary protein, urinary nitrites, bilirubin, red blood cell urinalysis, and the like.

With continued reference to FIG. 1, nutrient as used herein, includes any substance required by the human body to function. Nutrients may include carbohydrates, protein, lipids, vitamins, minerals, antioxidants, fatty acids, amino acids, and the like. Nutrients may include for example vitamins such as thiamine, riboflavin, niacin, pantothenic acid, pyridoxine, biotin, folate, cobalamin, Vitamin C, Vitamin A, Vitamin D, Vitamin E, and Vitamin K. Nutrients may include for example minerals such as sodium, chloride, potassium, calcium, phosphorous, magnesium, sulfur, iron, zinc, iodine, selenium, copper, manganese, fluoride, chromium, molybdenum, nickel, aluminum, silicon, vanadium, arsenic, and boron.

With continued reference to FIG. 1, nutrients may include extracellular nutrients that are free floating in blood and exist outside of cells. Extracellular nutrients may be located in serum. Nutrients may include intracellular nutrients which may be absorbed by cells including white blood cells and red blood cells.

With continued reference to FIG. 1, nutrient body measurement may include one or more blood test results that identify extracellular and intracellular levels of nutrients. Nutrient body measurement may include blood test results that identify serum, white blood cell, and red blood cell levels of nutrients. For example, nutrient body measurement may include serum, white blood cell, and red blood cell levels of micronutrients such as Vitamin A, Vitamin B1, Vitamin B2, Vitamin B3, Vitamin B6, Vitamin B12, Vitamin B5, Vitamin C, Vitamin D, Vitamin E, Vitamin K1, Vitamin K2, and folate.

With continued reference to FIG. 1, nutrient body measurement may include one or more blood test results that identify serum, white blood cell and red blood cell levels of nutrients such as calcium, manganese, zinc, copper, chromium, iron, magnesium, copper to zinc ratio, choline, inositol, carnitine, methylmalonic acid (MMA), sodium, potassium, asparagine, glutamine, serine, coenzyme q10, cysteine, alpha lipoic acid, glutathione, selenium, eicosapentaenoic acid (EPA), docosahexaenoic acid (DHA), docosapentaenoic acid (DPA), total omega-3, lauric acid, arachidonic acid, oleic acid, total omega 6, and omega 3 index.

With continued reference to FIG. 1, nutrient body measurement may include one or more salivary test results that identify levels of nutrients including any of the nutrients as described herein. Nutrient body measurement may include hair analysis of levels of nutrients including any of the nutrients as described herein.

With continued reference to FIG. 1, genetic as used herein, includes any inherited trait. Inherited traits may include genetic material contained with DNA including for example, nucleotides. Nucleotides include adenine (A), cytosine (C), guanine (G), and thymine (T). Genetic information may be contained within the specific sequence of an individual's nucleotides and sequence throughout a gene or DNA chain. Genetics may include how a particular genetic sequence may contribute to a tendency to develop a certain disease such as cancer or Alzheimer's disease.

With continued reference to FIG. 1, genetic body measurement may include one or more results from one or more blood tests, hair tests, skin tests, urine, amniotic fluid, buccal swabs and/or tissue test to identify a user's particular sequence of nucleotides, genes, chromosomes, and/or proteins. Genetic body measurement may include tests that example genetic changes that may lead to genetic disorders. Genetic body measurement may detect genetic changes such as deletion of genetic material or pieces of chromosomes that may cause Duchenne Muscular Dystrophy. Genetic body measurement may detect genetic changes such as insertion of genetic material into DNA or a gene such as the BRCA1 gene that is associated with an increased risk of breast and ovarian cancer due to insertion of 2 extra nucleotides. Genetic body measurement may include a genetic change such as a genetic substitution from a piece of genetic material that replaces another as seen with sickle cell anemia where one nucleotide is substituted for another. Genetic body measurement may detect a genetic change such as a duplication when extra genetic material is duplicated one or more times within a person's genome such as with Charcot-Marie Tooth disease type 1. Genetic body measurement may include a genetic change such as an amplification when there is more than a normal number of copies of a gene in a cell such as HER2 amplification in cancer cells. Genetic body measurement may include a genetic change such as a chromosomal translocation when pieces of chromosomes break off and reattach to another chromosome such as with the BCR-ABL1 gene sequence that is formed when pieces of chromosome 9 and chromosome 22 break off and switch places. Genetic body measurement may include a genetic change such as an inversion when one chromosome experiences two breaks and the middle piece is flipped or inverted before reattaching. Genetic body measurement may include a repeat such as when regions of DNA contain a sequence of nucleotides that repeat a number of times such as for example in Huntington's disease or Fragile X syndrome. Genetic body measurement may include a genetic change such as a trisomy when there are three chromosomes instead of the usual pair as seen with Down syndrome with a trisomy of chromosome 21, Edwards syndrome with a trisomy at chromosome 18 or Patau syndrome with a trisomy at chromosome 13. Genetic body measurement may include a genetic change such as monosomy such as when there is an absence of a chromosome instead of a pair, such as in Turner syndrome.

With continued reference to FIG. 1, genetic body measurement may include an analysis of COMT gene that is responsible for producing enzymes that metabolize neurotransmitters. Genetic body measurement may include an analysis of DRD2 gene that produces dopamine receptors in the brain. Genetic body measurement may include an analysis of ADRA2B gene that produces receptors for noradrenaline. Genetic body measurement may include an analysis of 5-HTTLPR gene that produces receptors for serotonin. Genetic body measurement may include an analysis of BDNF gene that produces brain derived neurotrophic factor. Genetic body measurement may include an analysis of 9p21 gene that is associated with cardiovascular disease risk. Genetic body measurement may include an analysis of APOE gene that is involved in the transportation of blood lipids such as cholesterol. Genetic body measurement may include an analysis of NOS3 gene that is involved in producing enzymes involved in regulating vaso-dilation and vaso-constriction of blood vessels.

With continued reference to FIG. 1, genetic body measurement may include ACE gene that is involved in producing enzymes that regulate blood pressure. Genetic body measurement may include SLCO1B1 gene that directs pharmaceutical compounds such as statins into cells. Genetic body measurement may include FUT2 gene that produces enzymes that aid in absorption of Vitamin B12 from digestive tract. Genetic body measurement may include MTHFR gene that is responsible for producing enzymes that aid in metabolism and utilization of Vitamin B9 or folate. Genetic body measurement may include SHMT1 gene that aids in production and utilization of Vitamin B9 or folate. Genetic body measurement may include MTRR gene that produces enzymes that aid in metabolism and utilization of Vitamin B12. Genetic body measurement may include MTR gene that produces enzymes that aid in metabolism and utilization of Vitamin B12. Genetic body measurement may include FTO gene that aids in feelings of satiety or fulness after eating. Genetic body measurement may include MC4R gene that aids in producing hunger cues and hunger triggers. Genetic body measurement may include APOA2 gene that directs body to produce ApoA2 thereby affecting absorption of saturated fats. Genetic body measurement may include UCP1 gene that aids in controlling metabolic rate and thermoregulation of body. Genetic body measurement may include TCF7L2 gene that regulates insulin secretion. Genetic body measurement may include AMY1 gene that aids in digestion of starchy foods. Genetic body measurement may include MCM6 gene that controls production of lactase enzyme that aids in digesting lactose found in dairy products. Genetic body measurement may include BCMO1 gene that aids in producing enzymes that aid in metabolism and activation of Vitamin A. Genetic body measurement may include SLC23A1 gene that produce and transport Vitamin C. Genetic body measurement may include CYP2R1 gene that produce enzymes involved in production and activation of Vitamin D. Genetic body measurement may include GC gene that produce and transport Vitamin D. Genetic body measurement may include CYP1A2 gene that aid in metabolism and elimination of caffeine. Genetic body measurement may include CYP17A1 gene that produce enzymes that convert progesterone into androgens such as androstenedione, androstendiol, dehydroepiandrosterone, and testosterone.

With continued reference to FIG. 1, genetic body measurement may include CYP19A1 gene that produce enzymes that convert androgens such as androstenedione and testosterone into estrogens including estradiol and estrone. Genetic body measurement may include SRD5A2 gene that aids in production of enzymes that convert testosterone into dihydrotestosterone. Genetic body measurement may include UFT2B17 gene that produces enzymes that metabolize testosterone and dihydrotestosterone. Genetic body measurement may include CYP1A1 gene that produces enzymes that metabolize estrogens into 2 hydroxy-estrogen. Genetic body measurement may include CYP1B1 gene that produces enzymes that metabolize estrogens into 4 hydroxy-estrogen. Genetic body measurement may include CYP3A4 gene that produces enzymes that metabolize estrogen into 16 hydroxy-estrogen. Genetic body measurement may include COMT gene that produces enzymes that metabolize 2 hydroxy-estrogen and 4 hydroxy-estrogen into methoxy estrogen. Genetic body measurement may include GSTT1 gene that produces enzymes that eliminate toxic by-products generated from metabolism of estrogens. Genetic body measurement may include GSTM1 gene that produces enzymes responsible for eliminating harmful by-products generated from metabolism of estrogens. Genetic body measurement may include GSTP1 gene that produces enzymes that eliminate harmful by-products generated from metabolism of estrogens. Genetic body measurement may include SOD2 gene that produces enzymes that eliminate oxidant by-products generated from metabolism of estrogens.

With continued reference to FIG. 1, metabolic, as used herein, includes any process that converts food and nutrition into energy. Metabolic may include biochemical processes that occur within the body. Metabolic body measurement may include blood tests, hair tests, skin tests, amniotic fluid, buccal swabs and/or tissue test to identify a user's metabolism. Metabolic body measurement may include blood tests that examine glucose levels, electrolytes, fluid balance, kidney function, and liver function. Metabolic body measurement may include blood tests that examine calcium levels, albumin, total protein, chloride levels, sodium levels, potassium levels, carbon dioxide levels, bicarbonate levels, blood urea nitrogen, creatinine, alkaline phosphatase, alanine amino transferase, aspartate amino transferase, bilirubin, and the like.

With continued reference to FIG. 1, metabolic body measurement may include one or more blood, saliva, hair, urine, skin, and/or buccal swabs that examine levels of hormones within the body such as 11-hydroxy-androstereone, 11-hydroxy-etiocholanolone, 11-keto-androsterone, 11-keto-etiocholanolone, 16 alpha-hydroxyestrone, 2-hydroxyestrone, 4-hydroxyestrone, 4-methoxyestrone, androstanediol, androsterone, creatinine, DHEA, estradiol, estriol, estrone, etiocholanolone, pregnanediol, pregnanestriol, specific gravity, testosterone, tetrahydrocortisol, tetrahydrocrotisone, tetrahydrodeoxycortisol, allo-tetrahydrocortisol.

With continued reference to FIG. 1, metabolic body measurement may include one or more metabolic rate test results such as breath tests that may analyze a user's resting metabolic rate or number of calories that a user's body burns each day rest. Metabolic body measurement may include one or more vital signs including blood pressure, breathing rate, pulse rate, temperature, and the like. Metabolic body measurement may include blood tests such as a lipid panel such as low density lipoprotein (LDL), high density lipoprotein (HDL), triglycerides, total cholesterol, ratios of lipid levels such as total cholesterol to HDL ratio, insulin sensitivity test, fasting glucose test, Hemoglobin A1C test, adipokines such as leptin and adiponectin, neuropeptides such as ghrelin, pro-inflammatory cytokines such as interleukin 6 or tumor necrosis factor alpha, anti-inflammatory cytokines such as interleukin 10, markers of antioxidant status such as oxidized low-density lipoprotein, uric acid, paraoxonase 1. Persons skilled in the art, upon reviewing the entirety of this disclosure, will be aware of various additional examples of physiological state data that may be used consistently with descriptions of systems and methods as provided in this disclosure.

With continued reference to FIG. 1, physiological data may be obtained from a physically extracted sample. A “physical sample” as used in this example, may include any sample obtained from a human body of a user. A physical sample may be obtained from a bodily fluid and/or tissue analysis such as a blood sample, tissue, sample, buccal swab, mucous sample, stool sample, hair sample, fingernail sample and the like. A physical sample may be obtained from a device in contact with a human body of a user such as a microchip embedded in a user's skin, a sensor in contact with a user's skin, a sensor located on a user's tooth, and the like. Physiological data may be obtained from a physically extracted sample. A physical sample may include a signal from a sensor configured to detect physiological data of a user and record physiological data as a function of the signal. A sensor may include any medical sensor and/or medical device configured to capture sensor data concerning a patient, including any scanning, radiological and/or imaging device such as without limitation x-ray equipment, computer assisted tomography (CAT) scan equipment, positron emission tomography (PET) scan equipment, any form of magnetic resonance imagery (MRI) equipment, ultrasound equipment, optical scanning equipment such as photo-plethysmographic equipment, or the like. A sensor may include any electromagnetic sensor, including without limitation electroencephalographic sensors, magnetoencephalographic sensors, electrocardiogramsors, electromyographic sensors, or the like. A sensor may include a temperature sensor. A sensor may include any sensor that may be included in a mobile device and/or wearable device, including without limitation a motion sensor such as an inertial measurement unit (IMU), one or more accelerometers, one or more gyroscopes, one or more magnetometers, or the like. At least a wearable and/or mobile device sensor may capture step, gait, and/or other mobility data, as well as data describing activity levels and/or physical fitness. At least a wearable and/or mobile device sensor may detect heart rate or the like. A sensor may detect any hematological parameter including blood oxygen level, pulse rate, heart rate, pulse rhythm, blood sugar, and/or blood pressure. A sensor may be configured to detect internal and/or external biomarkers and/or readings. A sensor may be a part of system 100 or may be a separate device in communication with system 100.

With continued reference to FIG. 1, computing device 104 is configured to receive condition state training data 112. “Condition state training data,” as used in this disclosure, is training data that contains a plurality of physiological data sets and a plurality of correlated condition state label. Training data, as used in this disclosure, is data containing correlations that a machine-learning process may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine-learning processes as described in further detail below. Training data may be formatted and/or organized by categories of data elements, for instance by associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements; for instance, and without limitation, training data may be provided in fixed-length formats, formats linking positions of data to categories such as comma-separated value (CSV) formats and/or self-describing formats such as extensible markup language (XML), enabling processes or devices to detect categories of data.

Alternatively or additionally, and still referring to FIG. 1, training data may include one or more elements that are not categorized; that is, training data may not be formatted or contain descriptors for some elements of data. Machine-learning algorithms and/or other processes may sort training data according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below. Training data used by computing device 104 may correlate any input data as described in this disclosure to any output data as described in this disclosure.

With continued reference to FIG. 1, a “condition state label,” as used in this disclosure, is an element of data identifying and/or describing a current, incipient, or probable future medical condition affecting a human being; medical condition may include a particular disease, one or more symptoms associated with a syndrome, a syndrome, and/or any other measure of current or future health and/or heathy aging. A condition state label 116 may identify a disease including any condition that impairs the normal function of the human body. A condition state label 116 may identify the absence of a disease or condition. For example, a condition state label 116 may identify a disease such as an infection, or a genetic disease. In yet another non-limiting example, a condition state label 116 may identify that a user is free and clear of disease. A condition state label 116 may identify an acquired disease such as one that begins at some point during one's lifetime, as opposed to disease already present at birth. For example, a condition state label 116 may identify an acquired disease such as viral cardiomyopathy. A condition state label 116 may identify a congenital disease that may be present at birth such as a baby born with human immune deficiency virus (HIV). A congenial disease may include an inherited genetic disease or disorder such as for example cystic fibrosis, marfan syndrome, fragile X syndrome, or hemochromatosis. A condition state label 116 may identify an acute disease such as one that is of a short-term nature such as a urinary tract infection (UTI). A condition state label 116 may identify a chronic condition and/or chronic disease such as hypertension. A condition state label 116 may identify a genetic disorder and/or genetic disease that may be caused by one or more genetic mutations. For example, a condition state label 116 may identify a genetic disease such as Huntington's disease. A condition state label 116 may identify a hereditary and/or inherited disease such as familial hypercholesterolemia. A condition state label 116 may identify an iatrogenic disease caused by medical intervention such as a side effect of a treatment or as an inadvertent outcome. For example, a condition state label 116 may identify a prescription drug adverse effect such as an antibiotic that causes excessive diarrhea or vertigo experienced as a side effect from brain surgery. A condition state label 116 may identify idiopathic disease such as disease having an unknown cause or source. For example, a condition state label 116 may identify an idiopathic disease such as multiple sclerosis or diabetes mellitus type 1. A condition state label 116 may identify an incurable disease such as a disease that is terminal or a disease that has to be treated for the rest of a user's life. A condition state label 116 may identify primary disease that is due to a root cause illness such as a bacterial infection. A condition state label 116 may identify secondary disease that is a sequela and/or complication of a prior, causal disease such as for example, a bacterial infection that develops at the site of a burn. A condition state label 116 may identify a terminal disease such as a disease that is expected to have the inevitable result of death. A condition state label 116 may identify a disorder that may include a functional abnormality and/or disturbance. For example, a condition state label 116 may identify a mental disorder, physical disorder, genetic disorder, emotional disorder, behavior disorder, functional disorder and the like. A condition state label 116 may identify a medical condition that includes all diseases, lesions, disorders, and/or nonpathological conditions that require medical treatment. A condition state label 116 may identify a syndrome that may include an association of several medical signs and symptoms that occur together. For example, a condition state label 116 may identify a syndrome such as Down syndrome, Parkinsonian syndrome, acute coronary syndrome and the like. A condition state label 116 may identify predisease, which includes any subclinical and/or prodromal presentation of a disease. For example, a condition state label 116 may identify predisease such as prediabetes or prehypertension. A condition state label 116 may identify an affected body system such as the renal system when a disease or condition affects the kidneys.

With continued reference to FIG. 1, computing device 104 may receive condition state training data 112 generated from one or more user entries. In an embodiment, computing device 104 may receive previously collected user data to generate condition state training data that includes a plurality of physiological data obtained from user entries and a plurality of correlated user condition state labels obtained from user entries. In an embodiment, one or more biological extraction 108, one or more elements of user physiological data, and/or one or more condition state label 116 may be stored in a user database 120. User database 120 may be implemented, without limitation, as a relational database, a key-value retrieval datastore such as a NOSQL database, or any other form or structure for use as a datastore that a person skilled in the art would recognize as suitable upon review of the entirety of this disclosure. Computing device 104 may retrieve previous user entries to generate condition state training data 112 based on one or more previous user entries.

With continued reference to FIG. 1, computing device 104 is configured to generate a condition state model utilizing one or more machine-learning processes. Condition state model is a machine-learning model, which as used herein, is a mathematical representation of a relationship between inputs and outputs, as generated using any machine-learning process including without limitation any process as described above, and stored in memory; an input is submitted to a machine-learning model once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model may be generated by creating an artificial neural network, such as a convolutional neural network comprising an input layer of nodes, one or more intermediate layers, and an output layer of nodes. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

With continued reference to FIG. 1, a machine learning process, also referred to as a machine-learning algorithm, is a process that automatedly uses training data and/or a training set as described above to generate an algorithm that will be performed by a computing device 104 and/or module to produce outputs given data provided as inputs; this is in contrast to a non-machine learning software program where the commands to be executed are determined in advance by a user and written in a programming language.

Continuing to refer to FIG. 1, machine-learning algorithms may be implemented using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure,

Still referring to FIG. 1, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

With continued reference to FIG. 1, models may be generated using alternative or additional artificial intelligence methods, including without limitation by creating an artificial neural network, such as a convolutional neural network comprising an input layer of nodes, one or more intermediate layers, and an output layer of nodes. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. This network may be trained using training data.

Still referring to FIG. 1, machine-learning algorithms may include supervised machine-learning algorithms. Supervised machine learning algorithms, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised machine-learning process may include a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data. Persons skilled in the art, upon reviewing the entirety of this disclosure, will be aware of various possible variations of supervised machine learning algorithms that may be used to determine relation between inputs and outputs.

With continued reference to FIG. 1, supervised machine-learning processes may include classification algorithms, defined as processes whereby a computing device 104 derives, from training data, a model for sorting inputs into categories or bins of data. Classification may be performed using, without limitation, linear classifiers such as without limitation logistic regression and/or naive Bayes classifiers, nearest neighbor classifiers including without limitation k-nearest neighbors classifiers, support vector machines, decision trees, boosted trees, random forest classifiers, and/or neural network-based classifiers.

Still referring to FIG. 1, machine learning processes may include unsupervised processes. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like. Unsupervised machine-learning algorithms may include, without limitation, clustering algorithms and/or cluster analysis processes, such as without limitation hierarchical clustering, centroid clustering, distribution clustering, clustering using density models, subspace models, group models, graph-based models, signed graph models, neural models, or the like. Unsupervised learning may be performed by neural networks and/or deep learning protocols as described above.

With continued reference to FIG. 1, computing device 104 uses an element of user physiological data and condition state training data 112, in combination with a first machine-learning algorithm to utilize physiological data as inputs and output condition state label 116. First machine-learning algorithm includes any of the machine-learning algorithms as described herein. In an embodiment, one or more machine-learning processes such as condition state model, condition state label 116, and/or condition state training data may be stored in machine-learning database 124. Machine-learning database 124 may be implemented as any data structure suitable for use as user database 120 as described above in more detail.

With continued reference to FIG. 1, computing device 104 is configured to calculate a condition state label 116 for an element of user physiological data using a condition state model 128. Condition state label 116 includes any of the condition state label 116 as described above. A “condition state model,” as used in this disclosure, is a machine-learning model that utilizes physiological data as inputs and outputs condition state label. Generating a condition state model may include performing a series of one or more calculations, algorithms, and/or equations. Condition state model may include any of the machine-learning models as described herein. In an embodiment, condition state model may include a classification algorithm, defined as processes whereby a computing device 104 derives, from training data, a model for sorting inputs into categories or bins of data. Classification may be performed using, without limitation, linear classifiers such as without limitation logistic regression and/or naive Bayes classifiers, nearest neighbor classifiers including without limitation k-nearest neighbors classifiers, support vector machines, decision trees, boosted trees, random forest classifiers, and/or neural network-based classifiers.

With continued reference to FIG. 1, system 100 may include a remote device 132. Remote device 132 may include without limitation, a display in communication with computing device 104, where a display may include any display as described herein. Remote device 132 may include an additional computing device, such as a mobile device, laptop, desktop, computer and the like. Remote device 132 may be configured to transmit and/or receive one or more inputs from computing device 104 utilizing any network methodology as described herein. Remote device 132 may be operated by a user which may include any human subject. Remote device 132 may be operated by an informed advisor, which may include any healthcare provider such as for example, a functional medicine physician, a nurse practitioner, a yoga teacher, a fitness instructor, a health coach, a reiki master, a massage therapist, an acupuncturest and the like.

With continued reference to FIG. 1, computing device 104 may receive from a remote device 132 a description of a current condition state 136. A “current condition state,” as used in this disclosure, is data describing any current, incipient, or probable future medical condition that a user has been diagnosed with. A current condition state 136 may include a description of a current disease state such as rheumatoid arthritis that a use was diagnosed with three years back. A current condition state 136 may include a description of a probable future medical condition that a user may be diagnosed with in the future such as type 2 diabetes mellitus due to a user's current diagnosis of prediabetes. A current condition state may be generated by a user such as when a user enters one or more current condition state 136 that the user was previously diagnosed with and self-reports. A current condition state 136 may be generated by an informed advisor, such as when a user's functional medicine physician may generate a current condition state 136 at a remote device 132 and transmit the current condition state 136 to computing device 104. In an embodiment, a current condition state 136 may be stored within user database 120. Computing device 104 may utilize a description of a current condition state 136 received from a remote device 132 to calculate a condition state label 116 to contain a current condition state progression indicator 140. A “current condition state progression indicator,” as used in this disclosure, is a description of how far progressed a current condition state 136 is. Progression may include one or more classifications that indicate how advanced a particular condition state may be based on factors such as etiology, pathophysiology, and/or severity. Progression may indicate if a particular disease and/or condition is acute, such as when a disease may be short-lived such as the common cold. Progression may indicate if a particular disease and/or condition is chronic, such as a disease that lasts for an extended duration, such as more than six months. Progression may indicate a clinical disease, such as a disease that has clinical consequences. For example, acquired immune deficiency syndrome (AIDS), may be a clinical disease state of human immune deficiency virus (HIV). Progression may indicate a flare-up, such as when there is a recurrence of symptoms of a disease or condition, and/or when there is an onset of more severe symptoms. Progression may indicate a disease and/or condition whose typical natural course is worsening such as multiple sclerosis that is progressing to an extent where a user can no longer walk without the use of a cane. Progression may indicate a refractory disease, such as a disease that resists treatment. Progression may indicate a subclinical disease, such as a silent disease that occurs before symptoms are first noticed. Progression may indicate a terminal phase, such as a disease where a user will die soon, such as terminal cancer. Progression may indicate an extent of disease such as a localized disease that affects only one part of the body, such as athlete's foot or an eye infection. Progression may indicate disseminated disease that has spread to other areas of the body, such as cancer. Progression may indicate systemic disease, such as a disease that affects the entire body such as influenza or high blood pressure. Progression may indicate diseases that may be classified by involved organ system. Progression may indicate cause, such as a disease caused by lifestyle and behavioral choices. Progression may indicate infection rate of disease such as transmission of communicable disease. Computing device 104 may utilize a condition state label 116 containing a current condition state progression indicator 140 to select a treatment training set 152 and a treatment training model as described below in more detail.

With continued reference to FIG. 1, condition state progression indicator may be generated utilizing a machine-learning process. Computing device 104 may receive progression training data 144. “Progression training data,” as used in this disclosure, is training data that contains a plurality of physiological data sets and a plurality of correlated progression indicators. “Progression indicators” as used in this disclosure, are a description of how far progressed a current condition state 136 is. Progression indicators may include any data suitable for use as condition state progression indicator. Computing device 104 generates a progression model 148 using a machine-learning algorithm, the element of user physiological data, and the progression training data 144. Machine-learning algorithm includes any of the machine-learning algorithms as described herein. A “progression model,” as used in this disclosure, is a machine-learning model that utilizes physiological data as inputs and outputs progression indicators. A progression model 148 may include performing a series of one or more calculations, algorithms, and/or equations. Computing device 104 calculates a condition state progression indicator utilizing a progression model 148. Progression model 148 may be generated by calculating one or more machine-learning algorithms, including any of the machine-learning algorithms as described herein.

With continued reference to FIG. 1, computing device 104 is configured to select a treatment training set 152 and utilizing a condition state label 116. A “treatment training set,” as used in this disclosure, is a set of training data that contains a plurality of condition state labels 116 and a plurality of correlated treatments. A “treatment,” as used in this disclosure, is any process given, prescribed, and/or recommended for a condition which may be identified using any condition state label 116. A treatment may include a medication including for example any prescription and/or non-prescription medications such as vitamins, herbs, supplements, homeopathic remedies, nutraceuticals, minerals, cosmetics, prescription medications dispensed at a pharmacy, and the like. A treatment may include a program such as for example a fitness program containing recommended exercises and exercise routines. A treatment may include a meditation program such as for example a meditation practice to be implemented into a user's everyday life routine. A treatment may include a food program such as for example recommended foods to consume and not consume as well as one or more meal plans. A treatment may include one or more procedures including for example surgical and/or non-surgical procedures. A treatment may include a spiritual practice which may be associated with a particular religion such as Christianity for example. A treatment may include one or more psychological treatments including for example therapy sessions, behavior modifications, and counseling services.

With continued reference to FIG. 1, computing device 104 may select a treatment training set 152 and/or a treatment model 156 that corresponds to condition state label 116. A “treatment model,” as used in this disclosure, is a machine-learning model that utilizes a condition state label 116 as an input and outputs treatments. Generating a treatment model 156 may include performing a series of one or more calculations, algorithms, and/or equations. Treatment model 156 may include calculating one or more machine-learning algorithms, including any of the machine-learning algorithms as described above. In an embodiment, treatment training set 152 and/or a treatment model 156 may contain a label identifying which conditions, identified by condition state label 116, treatment training set 152 and/or treatment model 156 may be utilized for. Computing device 104 may identify treatment training set 152 and/or treatment model 156 intended for an output condition state label 116 and select a treatment training set 152 and/or treatment model 156 that hews most closely to a condition state label 116. For example, computing device 104 may identify a first treatment training set 152 and a first treatment model 156 that may be utilized for autoimmune conditions such as multiple sclerosis, ulcerative colitis, Crohn's disease, and Hashimoto's thyroiditis; and a second treatment training set 152 and a second treatment model 156 that may be utilized for ulcerative colitis. Computing device 104 may select second treatment training set 152 and second treatment model 156 for a condition state label 116 that contains ulcerative colitis, as second treatment training set 152 and second treatment model 156 are specifically intended for ulcerative colitis, while first treatment training set 152 and first treatment model 156 are intended more broadly for autoimmune conditions. In an embodiment, treatment training set 152 and treatment model 156 may be stored within machine-learning database 124. In an embodiment, treatment model may be precalculated and stored within a database and ready to be generated immediately.

With continued reference to FIG. 1, computing device 104 may receive a treatment response from a user. In some embodiments, computing device 104 may receive a treatment response from a remote device 132 of a user. For the purposes of this disclosure, a “treatment response” is data containing a user's opinion or feedback regarding a treatment. In some embodiments, treatment response may include information regarding how the user is feeling. In some embodiments, treatment response may include qualitative data. Qualitative data may include descriptions of the user's opinions or feedback regarding the treatment. Qualitative data may include descriptions of how the treatment makes the user feel. Qualitative data may include “ok,” “bad,” “great,” “good,” or longer thoughts, such as “I think the treatment has not benefited my condition,” “this treatment makes me feel lethargic,” and the like. In some embodiments, treatment response may include quantitative data. Quantitative data may include a user's rating, on a numerical scale, of how they feel, or, for example, their opinions on the treatment. Numerical scale may include any suitable numerical scale. Numerical scale may include, but is not limited to, −1 to 1, −100 to 100, 0 to 5, 0 to 10, 0 to 50, 0 to 100, a binary scale including just “0” and “1,” and the like.

With continued reference to FIG. 1, in some embodiments, computing device 104 may generate a treatment response form. For the purposes of this disclosure, a “treatment response form” is a prompt that is configured to solicit at least a treatment response from a user. Treatment response form may include a plurality of questions. For example, treatment response form may include questions such as “how does the treatment make you feel?”, “do you feel any improvement?”, “has your energy increased?”, “do you have any pain?”, and the like. In some embodiments, treatment response form may be transmitted to remote device 132. Treatment response form may include a series of ratings for a user to fill out. Treatment response form may include a space for a user to record how well they have been following a treatment. For example, treatment response form may allow for users to record missed days, deviations, issues, and the like.

With continued reference to FIG. 1, computing device 104 may receive a completed treatment response form from remote device 132. In some embodiments, computing device 104 may be configured to extract treatment responses from treatment response form. For example, any ratings or answers from treatment response form may be extracted as treatment responses.

With continued reference to FIG. 1, in some embodiments, computing device 104 may perform optical character recognition (OCR) on treatment response form or treatment responses. Optical character recognition or optical character reader (OCR) includes automatic conversion of images of written (e.g., typed, handwritten or printed text) into machine-encoded text. In some cases, recognition of at least a keyword from an image component may include one or more processes, including without limitation optical character recognition (OCR), optical word recognition, intelligent character recognition, intelligent word recognition, and the like. In some cases, OCR may recognize written text, one glyph or character at a time. In some cases, optical word recognition may recognize written text, one word at a time, for example, for languages that use a space as a word divider. In some cases, intelligent character recognition (ICR) may recognize written text one glyph or character at a time, for instance by employing machine learning processes. In some cases, intelligent word recognition (IWR) may recognize written text, one word at a time, for instance by employing machine learning processes.

Still referring to FIG. 1, in some cases OCR may be an “offline” process, which analyses a static document or image frame. In some cases, handwriting movement analysis can be used as input to handwriting recognition. For example, instead of merely using shapes of glyphs and words, this technique may capture motions, such as the order in which segments are drawn, the direction, and the pattern of putting the pen down and lifting it. This additional information can make handwriting recognition more accurate. In some cases, this technology may be referred to as “online” character recognition, dynamic character recognition, real-time character recognition, and intelligent character recognition.

Still referring to FIG. 1, in some cases, OCR processes may employ pre-processing of image component. Pre-processing process may include without limitation de-skew, de-speckle, binarization, line removal, layout analysis or “zoning,” line and word detection, script recognition, character isolation or “segmentation,” and normalization. In some cases, a de-skew process may include applying a transform (e.g., homography or affine transform) to image component to align text. In some cases, a de-speckle process may include removing positive and negative spots and/or smoothing edges. In some cases, a binarization process may include converting an image from color or greyscale to black-and-white (i.e., a binary image). Binarization may be performed as a simple way of separating text (or any other desired image component) from a background of image component. In some cases, binarization may be required for example if an employed OCR algorithm only works on binary images. In some cases. a line removal process may include removal of non-glyph or non-character imagery (e.g., boxes and lines). In some cases, a layout analysis or “zoning” process may identify columns, paragraphs, captions, and the like as distinct blocks. In some cases, a line and word detection process may establish a baseline for word and character shapes and separate words, if necessary. In some cases, a script recognition process may, for example in multilingual documents, identify script allowing an appropriate OCR algorithm to be selected. In some cases, a character isolation or “segmentation” process may separate signal characters, for example character-based OCR algorithms. In some cases, a normalization process may normalize aspect ratio and/or scale of image component.

Still referring to FIG. 1, in some embodiments an OCR process will include an OCR algorithm. Exemplary OCR algorithms include matrix matching process and/or feature extraction processes. Matrix matching may involve comparing an image to a stored glyph on a pixel-by-pixel basis. In some case, matrix matching may also be known as “pattern matching,” “pattern recognition,” and/or “image correlation.” Matrix matching may rely on an input glyph being correctly isolated from the rest of the image component. Matrix matching may also rely on a stored glyph being in a similar font and at a same scale as input glyph. Matrix matching may work best with typewritten text.

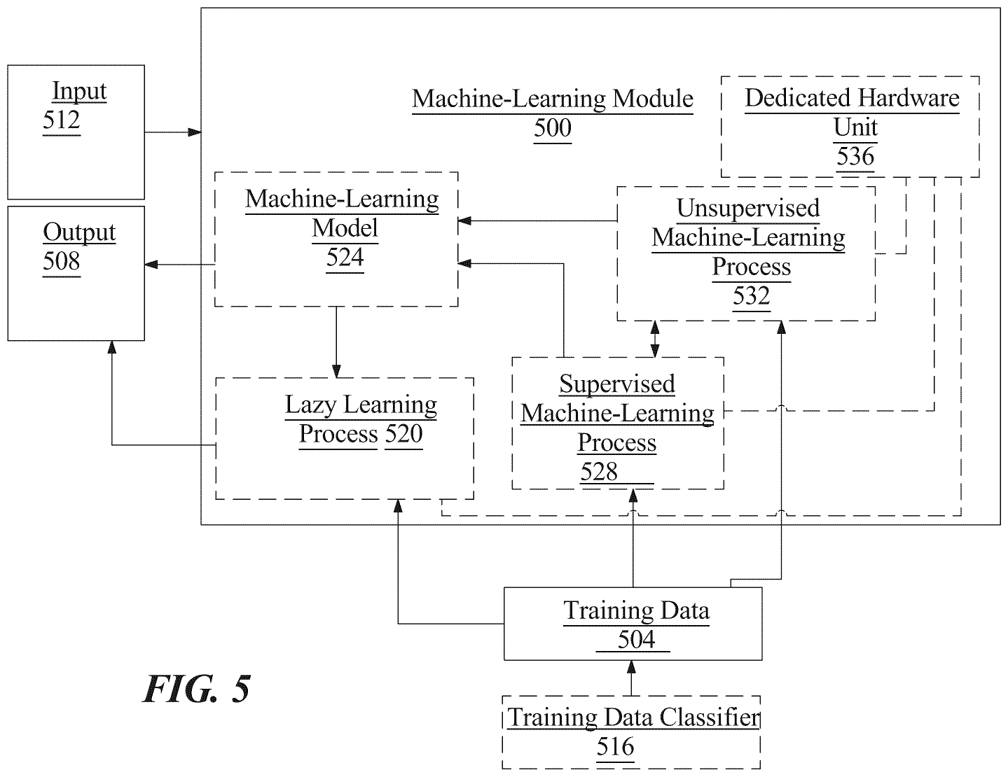

Still referring to FIG. 1, in some embodiments, an OCR process may include a feature extraction process. In some cases, feature extraction may decompose a glyph into features. Exemplary non-limiting features may include corners, edges, lines, closed loops, line direction, line intersections, and the like. In some cases, feature extraction may reduce dimensionality of representation and may make the recognition process computationally more efficient. In some cases, extracted feature can be compared with an abstract vector-like representation of a character, which might reduce to one or more glyph prototypes. General techniques of feature detection in computer vision are applicable to this type of OCR. In some embodiments, machine-learning process like nearest neighbor classifiers (e.g., k-nearest neighbors algorithm) can be used to compare image features with stored glyph features and choose a nearest match. OCR may employ any machine-learning process described in this disclosure, for example machine-learning processes described with reference to FIGS. 5-7. Exemplary non-limiting OCR software includes Cuneiform and Tesseract. Cuneiform is a multi-language, open-source optical character recognition system originally developed by Cognitive Technologies of Moscow, Russia. Tesseract is free OCR software originally developed by Hewlett-Packard of Palo Alto, California, United States.

Still referring to FIG. 1, in some cases, OCR may employ a two-pass approach to character recognition. Second pass may include adaptive recognition and use letter shapes recognized with high confidence on a first pass to recognize better remaining letters on the second pass. In some cases, two-pass approach may be advantageous for unusual fonts or low-quality image components where visual verbal content may be distorted. Another exemplary OCR software tool include OCRopus. OCRopus development is led by German Research Centre for Artificial Intelligence in Kaiserslautern, Germany. In some cases, OCR software may employ neural networks, for example neural networks as taught in reference to FIGS. 5-7.

Still referring to FIG. 1, in some cases, OCR may include post-processing. For example, OCR accuracy can be increased, in some cases, if output is constrained by a lexicon. A lexicon may include a list or set of words that are allowed to occur in a document. In some cases, a lexicon may include, for instance, all the words in the English language, or a more technical lexicon for a specific field. In some cases, an output stream may be a plain text stream or file of characters. In some cases, an OCR process may preserve an original layout of visual verbal content. In some cases, near-neighbor analysis can make use of co-occurrence frequencies to correct errors, by noting that certain words are often seen together. For example, “Washington, D.C.” is generally far more common in English than “Washington DOC.” In some cases, an OCR process may make us of a priori knowledge of grammar for a language being recognized. For example, grammar rules may be used to help determine if a word is likely to be a verb or a noun. Distance conceptualization may be employed for recognition and classification. For example, a Levenshtein distance algorithm may be used in OCR post-processing to further optimize results.

With continued reference to FIG. 1, computing device 104 may be configured to categorize treatment responses to one or more treatment response categories. A “treatment response category,” for the purposes of this disclosure, is an associative group of treatment responses. As a non-limiting example, qualitative treatment responses may be assigned to different treatment response categories corresponding to a numerical scale. In some embodiments, computing device 104 may use natural language processing (as disclosed throughout this disclosure) to categorize treatment responses to treatment response categories.

With continued reference to FIG. 1, computing device 104 may be configured to use a treatment response classifier to categorize treatment responses to one or more treatment response categories. Treatment response classifier may be consistent with any classifier described in this disclosure. Treatment response classifier may be trained using treatment response training data. Treatment response training data may be received from machine-learning database 124. Treatment response training data may include a plurality of treatment responses correlated to treatment response categories. Treatment response categories may include, for example, “good,” “bad,” and “neutral.” In some embodiments, treatment response categories may include the target of the treatment response, such as “financial,” “time,” “health,” and the like.

With continued reference to FIG. 1, treatment response form may be periodically sent to or displayed on remote device 132. Treatment response form may be sent to or displayed on remote device 132 on a set periodic basis. This may allow for a user's progress and satisfaction to be periodically checked. This could be used to update a treatment to best suit a user. In some embodiments, treatment response form may prompt a user to provide updated user data, such as current condition state 136. As non-limiting examples, treatment response form may be sent twice a week, weekly, monthly, monthly, seasonally, or the like.

With continued reference to FIG. 1, computing device 104 may be configured to generate a treatment response score as a function of treatment response. In some embodiments, treatment response score may include a numerical value or a percentage. In some embodiments, treatment response score may be generated using treatment responses and treatment response categories. For example, in some embodiments, where the treatment response categories represent quantitative categorizations of treatment responses, the number of treatment responses in each category may be used to determine treatment response score. In some embodiments, treatment response score may be calculated as a percentage of treatment responses that are categorized as “good.” In some embodiments, treatment response score may be calculated as a percentage of treatment responses that are categorized as “bad.”

With continued reference to FIG. 1, computing device 104 may generate treatment response score using a score machine-learning model. Score machine-learning model may be consistent with any machine-learning model disclosed as part of this disclosure. Score machine-learning model may be trained using score training data. Score training data may be retrieved from machine-learning database 124. Score training data may include treatment responses correlated to treatment response scores. Score training data may include treatment responses and associated treatment response categories correlated to treatment response scores.

With continued reference to FIG. 1, computing device 104 may be configured to generate a notification as a function of treatment response score. A “notification,” for the purposes of this disclosure is a brief communication designed to bring information to the attention of a user. A notification may include a notification on a smartphone, smart watch, or the like. Notification may include a text message, email, chat, and the like. In some embodiments, computing device 104 may be configured to generate notification when treatment response score crosses a treatment response score threshold. Treatment response score threshold may represent a score that signifies that a treatment is not properly benefiting or satisfying a user. In some embodiments, if a treatment response score falls fellow a treatment response score threshold, then computing device may automatically generate and transmit notification. In some embodiments, transmitting notification may include transmitting notification to remote device 132.

With continued reference to FIG. 1, notification may include a healthcare notification. A “healthcare notification,” for the purposes of this disclosure, is a notification that suggests that a user meet with a healthcare professional. As a non-limiting example, computing device 104 may select a healthcare professional as a function of a treatment category. Treatment categories are disclosed further throughout this disclosure. In some embodiments, computing device 104 may use a lookup table to select healthcare professional, wherein the lookup table comprises treatment categories associated with healthcare professionals. In some embodiments, computing device may generate healthcare notification as a function of comparing treatment response score to treatment response score threshold. In some embodiments, computing device 104 may be configured to insert contact information of healthcare professional into healthcare notification.

With continued reference to FIG. 1, notification may include a progress notification. A “progress notification,” for the purposes of this disclosure, is a notification that includes information regarding the users progress in a treatment or lack thereof. Progress notification may be generated as a function of a treatment and treatment response. In some embodiments, treatment response or updated user data may be used to determine a user's progress in treatment and generate progress notification.

With continued reference to FIG. 1, notification may include an encouragement notification. An “encouragement notification,” for the purposes of this disclosure, is a notification that encourages a user to stick to a treatment. In some embodiments, computing device 104 may generate encouragement notifications as a function of a treatment response or treatment response score. In some embodiments, computing device may generate encouragement notification as a function of comparing treatment response score to treatment response score threshold. In some embodiments, if treatment response score falls below treatment response score threshold, computing device 104 may automatically generate encouragement notification. In some embodiments, encouragement notification may be generated periodically, such as, for example, every day, every 3 days, every week, every other week, every month, and the like. Encouragement notification may include content that encourages a user to follow their treatment, for example, encouragement notification may tell a user the benefits of the treatment.

With continued reference to FIG. 1, in some embodiments, encouragement notification may be generated using generative AI. In some embodiments, generative AI may include a large language model (LLM). A “large language model,” as used herein, is a deep learning algorithm that can recognize, summarize, translate, predict and/or generate text and other content based on knowledge gained from massive datasets. Large language model may be trained on large sets of data; for example, training sets may include greater than 1 million words. Training sets may be drawn from diverse sets of data such as, as non-limiting examples, novels, blog posts, articles, emails, and the like. In some embodiments, training sets may include a variety of subject matters, such as, as nonlimiting examples, medical tests, romantic ballads, beat poetry, emails, advertising documents, newspaper articles, and the like. In some embodiments, training sets may be drawn from health blogs, cooking blogs, fitness advice websites, and the like.

With continued reference to FIG. 1, in some embodiments, LLM may be generally trained. For the purposes of this disclosure, “generally trained” means that LLM is trained on a general training set comprising a variety of subject matters, data sets, and fields. In some embodiments, LLM may be initially generally trained. In some embodiments, for the purposes of this disclosure, LLM may be specifically trained. For the purposes of this disclosure, “specifically trained” means that LLM is trained on a specific training set, wherein the specific training set includes data including specific correlations for LLM to learn. As a non-limiting example, LLM may be generally trained on a general training set, then specifically trained on a specific training set. As a non-limiting example, specific training set may include textual works 108. As a non-limiting example, specific training set may include scholastic works. As a non-limiting example, specific training set may include information from machine-learning database 124 and/or user database 120 and/or expert knowledge database 164.

With continued reference to FIG. 1, LLM, in some embodiments, may include Generative Pretrained Transformer (GPT), GPT-2, GPT-3, GPT-4, and the like. GPT, GPT-2, GPT-3, and GPT-4 are products of Open AI Inc., of San Francisco, CA. LLM may include a text prediction based algorithm configured to receive an article and apply a probability distribution to the words already typed in a sentence to work out the most likely word to come next in augmented articles. For example, if the words already typed are “Nice to meet”, then it is highly likely that the word “you” will come next. LLM may output such predictions by ranking words by likelihood or a prompt parameter. For the example given above, the LLM may score “you” as the most likely, “your” as the next most likely, “his” or “her” next, and the like. LLM may include an encoder component and a decoder component.

Still referring to FIG. 1, LLM may include a transformer architecture. In some embodiments, encoder component of LLM may include transformer architecture. A “transformer architecture,” for the purposes of this disclosure is a neural network architecture that uses self-attention and positional encoding. Transformer architecture may be designed to process sequential input data, such as natural language, with applications towards tasks such as translation and text summarization. Transformer architecture may process the entire input all at once. “Positional encoding,” for the purposes of this disclosure, refers to a data processing technique that encodes the location or position of an entity in a sequence. In some embodiments, each position in the sequence may be assigned a unique representation. In some embodiments, positional encoding may include mapping each position in the sequence to a position vector. In some embodiments, trigonometric functions, such as sine and cosine, may be used to determine the values in the position vector. In some embodiments, position vectors for a plurality of positions in a sequence may be assembled into a position matrix, wherein each row of position matrix may represent a position in the sequence.

With continued reference to FIG. 1, LLM and/or transformer architecture may include an attention mechanism. An “attention mechanism,” as used herein, is a part of a neural architecture that enables a system to dynamically quantify the relevant features of the input data. In the case of natural language processing, input data may be a sequence of textual elements. It may be applied directly to the raw input or to its higher-level representation.

With continued reference to FIG. 1, an attention mechanism may represent an improvement over a limitation of the Encoder-Decoder model. The encoder-decider model encodes the input sequence to one fixed length vector from which the output is decoded at each time step. This issue may be seen as a problem when decoding long sequences because it may make it difficult for the neural network to cope with long sentences, such as those that are longer than the sentences in the training corpus. Applying an attention mechanism, LLM may predict the next word by searching for a set of position in a source sentence where the most relevant information is concentrated. LLM may then predict the next word based on context vectors associated with these source positions and all the previously generated target words, such as textual data of a dictionary correlated to a prompt in a training data set. A “context vector,” as used herein, are fixed-length vector representations useful for document retrieval and word sense disambiguation.

Still referring to FIG. 1, an attention mechanism may include generalized attention self-attention, multi-head attention, additive attention, global attention, and the like. In generalized attention, when a sequence of words or an image is fed to LLM, it may verify each element of the input sequence and compare it against the output sequence. Each iteration may involve the mechanism's encoder capturing the input sequence and comparing it with each element of the decoder's sequence. From the comparison scores, the mechanism may then select the words or parts of the image that it needs to pay attention to. In self-attention, LLM may pick up particular parts at different positions in the input sequence and over time compute an initial composition of the output sequence. In multi-head attention, LLM may include a transformer model of an attention mechanism. Attention mechanisms, as described above, may provide context for any position in the input sequence. For example, if the input data is a natural language sentence, the transformer does not have to process one word at a time. In multi-head attention, computations by LLM may be repeated over several iterations, each computation may form parallel layers known as attention heads. Each separate head may independently pass the input sequence and corresponding output sequence element through a separate head. A final attention score may be produced by combining attention scores at each head so that every nuance of the input sequence is taken into consideration. In additive attention (Bahdanau attention mechanism), LLM may make use of attention alignment scores based on a number of factors. These alignment scores may be calculated at different points in a neural network. Source or input sequence words are correlated with target or output sequence words but not to an exact degree. This correlation may take into account all hidden states and the final alignment score is the summation of the matrix of alignment scores. In global attention (Luong mechanism), in situations where neural machine translations are required, LLM may either attend to all source words or predict the target sentence, thereby attending to a smaller subset of words.

With continued reference to FIG. 1, multi-headed attention in encoder may apply a specific attention mechanism called self-attention. Self-attention allows the models to associate each word in the input, to other words. So, as a non-limiting example, the LLM may learn to associate the word “you”, with “how” and “are”. It's also possible that LLM learns that words structured in this pattern are typically a question and to respond appropriately. In some embodiments, to achieve self-attention, input may be fed into three distinct fully connected layers to create query, key, and value vectors. The query, key, and value vectors may be fed through a linear layer; then, the query and key vectors may be multiplies using dot product matrix multiplication in order to produce a score matrix. The score matrix may determine the amount of focus for a word should be put on other words (thus, each word may be a score that corresponds to other words in the time-step). The values in score matrix may be scaled down. As a non-limiting example, score matrix may be divided by the square root of the dimension of the query and key vectors. In some embodiments, the softmax of the scaled scores in score matrix may be taken. The output of this softmax function may be called the attention weights. Attention weights may be multiplied by your value vector to obtain an output vector. The output vector may then be fed through a final linear layer.

With continued reference to FIG. 1, in order to use self-attention in a multi-headed attention computation, query, key, and value may be split into N vectors before applying self-attention. Each self-attention process may be called a “head.” Each head may produce an output vector and each output vector from each head may be concatenated into a single vector. This single vector may then be fed through the final linear layer discussed above. In theory, each head can learn something different from the input, therefore giving the encoder model more representation power.

With continued reference to FIG. 1, encoder of transformer may include a residual connection. Residual connection may include adding the output from multi-headed attention to the positional input embedding. In some embodiments, the output from residual connection may go through a layer normalization. In some embodiments, the normalized residual output may be projected through a pointwise feed-forward network for further processing. The pointwise feed-forward network may include a couple of linear layers with a ReLU activation in between. The output may then be added to the input of the pointwise feed-forward network and further normalized.

With continued reference to FIG. 1, transformer architecture may include a decoder. Decoder may a multi-headed attention layer, a pointwise feed-forward layer, one or more residual connections, and layer normalization (particularly after each sub-layer), as discussed in more detail above. In some embodiments, decoder may include two multi-headed attention layers. In some embodiments, decoder may be autoregressive. For the purposes of this disclosure, “autoregressive” means that the decoder takes in a list of previous outputs as inputs along with encoder outputs containing attention information from the input.

With continued reference to FIG. 1, in some embodiments, input to decoder may go through an embedding layer and positional encoding layer in order to obtain positional embeddings. Decoder may include a first multi-headed attention layer, wherein the first multi-headed attention layer may receive positional embeddings.

With continued reference to FIG. 1, first multi-headed attention layer may be configured to not condition to future tokens. As a non-limiting example, when computing attention scores on the word “am”, decoder should not have access to the word “fine” in “I am fine,” because that word is a future word that was generated after. The word “am” should only have access to itself and the words before it. In some embodiments, this may be accomplished by implementing a look-ahead mask. Look ahead mask is a matrix of the same dimensions as the scaled attention score matrix that is filled with “0s” and negative infinities. For example, the top right triangle portion of look-ahead mask may be filled with negative infinities. Look-ahead mask may be added to scaled attention score matrix to obtain a masked score matrix. Masked score matrix may include scaled attention scores in the lower-left triangle of the matrix and negative infinities in the upper-right triangle of the matrix. Then, when the softmax of this matrix is taken, the negative infinities will be zeroed out; this leaves zero attention scores for “future tokens.”

With continued reference to FIG. 1, second multi-headed attention layer may use encoder outputs as queries and keys and the outputs from the first multi-headed attention layer as values. This process matches the encoder's input to the decoder's input, allowing the decoder to decide which encoder input is relevant to put a focus on. The output from second multi-headed attention layer may be fed through a pointwise feedforward layer for further processing.

With continued reference to FIG. 1, the output of the pointwise feedforward layer may be fed through a final linear layer. This final linear layer may act as a classifier. This classifier may be as big as the number of classes that you have. For example, if you have 10,000 classes for 10,000 words, the output of that classifier will be of size 10,000. The output of this classifier may be fed into a softmax layer which may serve to produce probability scores between zero and one. The index may be taken of the highest probability score in order to determine a predicted word.

With continued reference to FIG. 1, decoder may take this output and add it to the decoder inputs. Decoder may continue decoding until a token is predicted. Decoder may stop decoding once it predicts an end token.

With continued reference to FIG. 1, in some embodiment, decoder may be stacked N layers high, with each layer taking in inputs from the encoder and layers before it. Stacking layers may allow LLM to learn to extract and focus on different combinations of attention from its attention heads.

With continued reference to FIG. 1, LLM may receive an input. Input may include a string of one or more characters. For example, input may include one or more words, a sentence, a paragraph, a thought, a query, and the like. In some embodiments, input may be received from remote device 132. In some embodiments, input may include treatment responses, treatment response scores, treatment response categories, treatments, treatment categories, user data, and the like.

With continued reference to FIG. 1, LLM may generate output. In some embodiments, LLM may include multiple sets of transformer architecture as described above. Output may include a textual output. A “textual output,” for the purposes of this disclosure is an output comprising a string of one or more characters. Textual output may include for example, a notification, such as an encouragement notification or text for the encouragement notification. Textual output may include for example, a notification, such as a healthcare notification or text for the healthcare notification. Textual output may include for example, a notification, such as a progress notification or text for the progress notification. Textual output may include for example, a notification, such as a support notification or text for the support notification

With continued reference to FIG. 1, LLM may be further trained using user feedback such as treatment responses. User feedback may include explicit feedback such as “5 starts” “great” “like,” and the like. In some embodiments, user feedback may be implicit. Implicit user feedback may be inferred from user actions. For example, if a certain amount of time passes before a user completes the task associated with the notification of encouragement, then processor may identify that as negative feedback. For example, if notification of encouragement results in completion of a treatment task, then processor may determine this to be positive feedback.

With continued reference to FIG. 1, computing device 104 may select a treatment training set 152 and/or a treatment model 156 by calculating a treatment category selector 160. A “treatment category selector,” as used in this disclosure, is a description of a particular category of treatment to be implemented and/or recommended for a particular condition identified from a condition state label 116. A category of treatment may include a class of treatments having shared characteristics. A category may include for example fitness treatments which may include further sub-categories such as cardiovascular fitness treatment, strength and toning fitness treatment, endurance fitness treatment, relaxation fitness treatment, meditative fitness treatment and the like. Other categories of treatments may include nutrition treatments, alternative medicine treatments, spiritual treatments, behavior modification treatments, prescription treatments, non-prescriptive treatments, coaching treatments, and the like. In an embodiment, certain conditions identified with condition state label 116 may respond more favorable to a particular treatment category selector 160 than another. For instance and without limitation, prediabetes may respond more favorably to condition treatment selectors that include fitness and nutritional treatment, while a gambling addiction may respond more favorably to behavior modification treatment and psychiatric treatment. Computing device 104 may calculate a treatment category selector 160 based on one or more inputs stored within user database 120 that may be generated by preferences input by a user and/or informed advisor. For example, a user may indicate that he prefers fitness treatments over nutritional treatments or that he does not enjoy specific forms of exercise that include biking and running.

With continued reference to FIG. 1, computing device 104 may calculate a treatment category selector 160. Computing device 104 may calculate a treatment category selector 160 by multiplying an approach factor multiplied by an implementation factor multiplied by a corrective factor. An output calculated treatment category selector 160 may be utilized to select a particular treatment training set 152 and/or treatment model 156. In an embodiment, a calculated treatment category selector 160 may output a numerical score with each factor utilized to calculate treatment category selector 160 being given an individual score that is multiplied together to produce a final output containing a calculated treatment category selector 160. The total final output calculated numerical value may be utilized to select a treatment training set 152 and/or treatment model 156 based on numerical ranges that may be assigned to treatment training set 152 and/or treatment model 156 based on expert input. Expert input may be provided and stored in an expert knowledge database 164. Expert knowledge database 164 may be implemented as any data structure suitable for use as user database 120 as described above. Expert knowledge database 164 may include expert input obtained from expert inputs such as top medical experts, journal articles, scientific studies and the like as described in more detail below. An “approach factor,” as used in this disclosure, is a factor that indicates an acceptable treatment approach by experts for a given condition identified with a condition state label 116. An approach factor 168 may include a standard endorsed by one or more treatment guidelines. This may include for example diagnostic and/or treatment processes that a healthcare provider may follow for a certain type of patient, illness, and/or clinical circumstance. A standard may be endorsed by one or more medical associations and/or organizations such as for example, THE INSTITUTE FOR FUNCTIONAL MEDICINE of Federal Way, Washington or THE AMERICAN ACADEMY OF ANTI-AGING MEDICINE of Boca Raton, Florida. A standard may be based on various approaches to medicine including for example conventional medicine approaches, functional medicine approaches, Western medical practices, Eastern medical practices, and/or any combination of the above. For instance and without limitation, a standard may include treating initial primary hypertension with lifestyle treatments including a fitness routine and nutritional treatments to remote excess sodium from one's diet. In yet another non-limiting example, a standard may include treating a systemic candida infection with anti-fungal medications in addition to dietary modifications that include initiating a grain free and refined sugar free diet. An “implementation factor,” as used in this disclosure, is a factor that indicates a user preference for different treatment practices. An implementation factor 172 may indicate if a user likes and/or dislikes different categories of treatments. For example, a user may prefer dietary treatments over prescriptive treatments or fitness treatments over medical procedures. One or more implementation factor 172 pertaining to a user may be received from a remote device 132 operated by a user and stored in user database 120. A “corrective factor,” as used in this disclosure, is a factor that indicates the intensity of a treatment. Intensity may include how soon a treatment needs to be implemented, how rigorous a course of treatment needs to be, the length of a particular treatment, and time commitment a user needs to devote to a treatment. For example, a corrective factor 176 may indicate that a treatment such as a surgical intervention needs to be performed immediately, while a treatment such as a fitness program will be implemented over the course of the next six months.

With continued reference to FIG. 1, computing device 104 may select a treatment training set 152 and a treatment model 156 based on user input. Computing device 104 may receive from a remote device 132 a user entry containing a current treatment input descriptor 180. A “current treatment input descriptor,” as used in this disclosure, is a description of any treatment a user may be currently practicing and/or prescribed. A current treatment input descriptor 180 may include a description of the treatment a user is currently practicing and has been prescribed, the frequency in which the user practices the treatment, the intensity, the time commitment, and the like. A current treatment input descriptor 180 may include a description of a prescription medication a user may be taking twice daily for hypertension. A current treatment input descriptor 180 may include a description of a fitness regimen that a user engages in to lose weight. A current treatment input descriptor 180 may include a description of a meditation practice that a user practices to combat a user's anxiety. Computing device 104 utilizes a current treatment input descriptor 180 to locate treatment training data and/or a treatment model 156. In an embodiment, treatment training data and/or a treatment model 156 may be categorized according to various classification schemes such as intensity levels, what condition state label 116 that are intended for, what role they play in treatment, when they should be implemented and the like. Computing device 104 may compare a current treatment input descriptor 180 to treatment training data and/or treatment model 156 to select related treatments, to select a related treatment, to select a higher or lower intensity treatment and the like. For instance and without limitation, a current treatment input descriptor 180 that includes a description of a user's current treatment that describes a user engages in yoga three times each week to improve a user's strength and flexibility may be utilized by computing device 104 to select a treatment training set 152 and/or treatment model 156 that contains a fitness treatment that includes yoga. In yet another non-limiting example, a current treatment input descriptor 180 that includes a user's current treatment as a prescription medication to reduce user's high cholesterol may be utilized by computing device 104 to select a treatment training set 152 and/or treatment model 156 that implements prescription medication treatments with dietary treatments.

With continued reference to FIG. 1, treatment model 156 may be adjusted as a function of treatment response, wherein the treatment response includes user feedback. Computing device 104 may use user feedback to train the treatment model 156. In some embodiments, if user feedback indicates that an output of treatment model 156 was “bad,” then that output and the corresponding input may be removed from training data used to train treatment model 156, and/or may be replaced with a value entered by, e.g., another user that represents an ideal output given the input the classifier originally received, permitting use in retraining, and adding to training data; in either case, treatment model 156 may be retrained with modified training data as described in further detail below. In some embodiments, training data of treatment model 156 may include user feedback.

With continued reference to FIG. 1, in some embodiments, an accuracy score may be calculated for treatment model 156 using user feedback. For the purposes of this disclosure, “accuracy score,” is a numerical value concerning the accuracy of a machine-learning model. For example, a plurality of user feedback scores may be averaged to determine an accuracy score. In some embodiments, a cohort accuracy score may be determined for particular cohorts of persons. For example, user feedback for users belonging to a particular cohort of persons may be averaged together to determine the cohort accuracy score for that particular cohort of persons, and used as described above. Accuracy score or another score as described above may indicate a degree of retraining needed for treatment model 156; computing device 104 may perform a larger number of retraining cycles for a higher number (or lower number, depending on a numerical interpretation used), and/or may collect more training data for such retraining, perform more training cycles, apply a more stringent convergence test such as a test requiring a lower mean squared error, and/or indicate to a user and/or operator that additional training data is needed.

With continued reference to FIG. 1, computing device 104 is configured to generate using a machine-learning algorithm, a condition state label 116 and the selected treatment training set 152 a treatment model 156.

With continued reference to FIG. 1, computing device 104 is configured to output a plurality of treatments utilizing the treatment model 156. In an embodiment, computing device 104 may display one or more treatments on a graphical user interface 184 located on computing device 104. Graphical user interface 184 may include without limitation a form or other graphical element having display fields, where one or more treatments may be displayed to a user. In an embodiment, computing device 104 may transmit a plurality of output treatments to a remote device 132 operated by a user and/or a user's informed advisor. For instance and without limitation, computing device 104 may transmit a plurality of output treatments to a user's functional medicine doctor or spiritual coach. In some embodiments, one or more treatments may be displayed on GUI 184 along with effectiveness information. As a non-limiting example, GUI 184 may display that with drug A, treatment will take X days, whereas with drug Y, treatment will take Y days. This may allow user to make a more informed choice as to what treatment to choose. In some embodiments, any side effects may be displayed for various drugs.

With continued reference to FIG. 1, computing device 104 may select a treatment from the plurality of output treatments by generating a loss function. Computing device 104 may utilize a loss function analysis utilizing linear regression to select a treatment from a plurality of output treatments. A “loss function,” as used in this disclosure, is an expression of an output of which an optimization algorithm minimizes to generate an optimal result. As a non-limiting example, computing device 104 may calculate variables based on a user input regarding various variables relating to treatments, calculate an output of mathematical expression using the variables, and select an element that produces an output having the lowest size, according to a given definition of “size,” of the sets of outputs representing each of the plurality of elements; size may, for instance, include absolute value, numerical size, or the like. Selection of different loss function may result in identification of different elements as generating minimal outputs; for instance, wherein a variable such as time commitment is associated in a first loss function with a large coefficient or weight, a variable such as cost having a small coefficient or weight may minimize the first loss function, whereas a second loss function where time commitment has a smaller coefficient but degree of variance from cost may produce a minimal output for a different variable having a larger coefficient for cost but more closely hewing to time commitment.

With continued reference to FIG. 1, mathematical expression and/or loss function may be generated using a machine learning to produce loss function: i.e., regression. Mathematical expression and/or loss function be user-specific, using a training set composed of previous user variables; which may be updated continuously. “User variables,” as used in this disclosure, are any current and/or previously entered user inputs regarding treatments. User variables may include inputs regarding how much money a user is willing to spend on treatment, how far a user is willing to travel for treatment, how long a user is willing to devote to treatment and the like. Mathematical expression and/or loss function may initially be seeded using one or more variables as described above. User may enter a new command changing mathematical expression, and then subsequent user selections may be used to generate a new training set to modify the new expression.

With continued reference to FIG. 1, mathematical expression may be adjusted as a function of treatment response. As a non-limiting example, if treatment response indicates that a user has been unable to stick to the treatment, then user variable corresponding to how long a user is willing to devote to treatment may be adjusted downwards. As a non-limiting example, if treatment response indicates that a user thinks that treatment has been too expensive, then user variable corresponding to how much a user is willing to spend on treatment may be adjusted downwards. After this adjustment, then the mathematical expression may be recalculated using the updated user variables.

With continued reference to FIG. 1, mathematical expression and/or loss function may be generated using machine learning using a multi-user training set. Training set may be created using data of a cohort of persons having similar demographic, religious, health, lifestyle characteristics, and/or variable rankings to user. This may alternatively or additionally be used to seed a mathematical expression and/or loss function for a user, which may be modified by further machine learning and/or regression using subsequent selection variables. Computing device 104 minimizes a loss function and selects a treatment from the plurality of output treatments for a user as a result of minimizing a loss function.

With continued reference to FIG. 1, computing device 104 may compare one or more user variables to a mathematical expression representing an optimal combination of user variable rankings. Mathematical expression may include a linear combination of variables, weighted by coefficients representing relative importance of each variable in selecting an optimal treatment. For instance, a variable such as treatment intensity may be multiplied by a first coefficient representing the importance of treatment intensity, a second variable such as cost may be multiplied by a second coefficient representing the importance of cost, a third variable may be multiplied by a third coefficient representing the importance of that variable; persons skilled in the art, upon reviewing the entirety of this disclosure, will be aware of different variables that may be weighted by various coefficients. Use of a linear combination is provided only as an illustrative example; other mathematical expressions may alternatively or additionally be used, including without limitation higher-order polynomial expressions or the like. Variables may include one or more user responses in regard to different treatment options. Variables may indicate for example, how much money a user is willing to spend on a treatment, how much time a user is willing to devote to a treatment, how complex of a treatment a user is willing to partake in, how intense of a treatment a user is interested in partaking, previous treatments, treatments that a user enjoyed, treatments that a user did not enjoy, treatments a user is able to partake in, treatments a user is unable to partake in, and the like.

With continued reference to FIG. 1, each user entry relating to a particular variable may be represented by a mathematical expression having the same form as mathematical expression; computing device 104 may compare the former to the latter using an error function representing average difference between the two mathematical expressions. Error function may, as a non-limiting example, be calculated using the average difference between coefficients corresponding to each input variable. A variable ranking having a mathematical expression minimizing the error function may be selected, as representing an optimal expression of relative importance of variables to a system or user. In an embodiment, error function and loss function calculations may be combined; for instance, a user entry ranking resulting in a minimal aggregate expression of error function and loss function, such as a simple addition, arithmetic mean, or the like of the error function with the loss function, may be selected, corresponding to an option that minimizes total variance from optimal variables while simultaneously minimizing a degree of variance from a set of priorities corresponding to additional variables. Coefficients of mathematical expression and/or loss function may be scaled and/or normalized; this may permit comparison and/or error function calculation to be performed without skewing by varied absolute quantities of numbers.

Still referring to FIG. 1, mathematical expression and/or loss function may be provided by receiving one or more user commands. For instance, and without limitation, a graphical user interface 184 may be provided to user with a set of sliders or other user inputs permitting a user to indicate relative and/or absolute importance of each variable containing a user entry ranking to the user. Sliders or other inputs may be initialized prior to user entry as equal or may be set to default values based on results of any machine-learning processes or combinations thereof as described in further detail below. One or more user entries relating to variables may be stored in user database 120.

With continued reference to FIG. 1, computing device 104 is configured to utilize generated outputs to updated training data sets and/or models contained within system 100. Computing device 104 may utilize an output plurality of treatments and a user biological extraction 108 to incorporate them into a subsequent training set. Computing device 104 may utilize an output condition state label 116 generated for an element of user physiological data, and an element of user physiological data to update condition state training data 112. Computing device 104 may incorporate one or more updated training sets and/or machine-learning models to be stored within machine-learning database 124.

With continued reference to FIG. 1, computing device 104 may generate a support network for a user. A “support network,” for the purposes of this disclosure, is a collection of other users that have commonality with a user. In some embodiments, computing device 104 may generate a support network for a user as a function of a treatment. For example, computing device 104 may identify other users that are undergoing the same treatment as user and assign them to the support network. In some embodiments, computing device 104 may generate a support network for a user as a function of a treatment category. For example, computing device 104 may identify other users that are undergoing the same category of treatment treatment as user and assign them to the support network. In some embodiments, user may be able to choose on remote device 132 whether they wish to join a support group. In some embodiments, users that do not wish to be in a support group may be excluded from the process above. With continued reference to FIG. 1, in some embodiments, computing device may generate a support notification. A “support notification’ is a notification regarding the joining of a support group. In some embodiments, support notification may tell a user to join a support group. In some embodiments, support notification may tell a user the other users who could be in their support group. In some embodiments, a support notification may be generated as a function of a treatment response score falling below a treatment response score threshold.

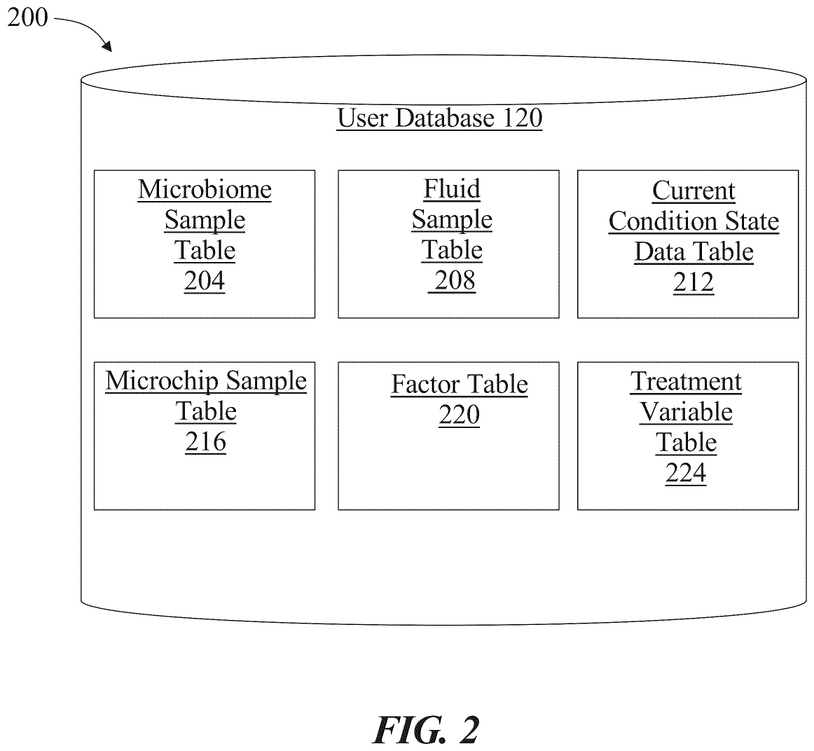

Referring now to FIG. 2, an exemplary embodiment of user database 120 is illustrated. User database 120 may be implemented as any data structure as described above in more detail. One or more tables contained within user database 120 may include microbiome sample table 204; microbiome sample table 204 may include one or more biological extraction 108 relating to the microbiome. For instance and without limitation, microbiome sample table 204 may include a physically extracted sample such as a stool sample analyzed for the presence of pathogenic species such as parasites and anaerobes. One or more tables contained within user database 120 may include fluid sample table 208; fluid sample table 208 may include one or more biological extraction 108 containing fluid samples. For instance and without limitation, fluid sample table 208 may include a urine sample analyzed for the presence or absence of glucose. One or more tables contained within user database 120 may include current condition state data table 212; current condition state 136 data table 212 may include one or more current conditions and/or treatments pertaining to a user. For instance and without limitation, current condition state data table 212 may include a user's previous diagnosis of a chronic medical condition such as rheumatoid arthritis and a current treatment the user engages in to control symptom such as a series of meditative poses. One or more tables contained within user database 120 may include microchip sample table 216; microchip sample table 216 may include one or more biological extractions obtained from a microchip. For instance and without limitation, microchip sample table 216 may include an intracellular nutrient level obtained from a microchip embedded under a user's skin. One or more tables contained within user database 120 may include factor table 220; factor table 220 may include one or more factors utilized to calculate a treatment category selector 160. For instance and without limitation, factor table 220 may include one or more approach factors 168, implementation factors 172, and/or corrective factors 176. One or more tables contained within user database 120 may include treatment variable table 224; treatment variable table 224 may include one or more user responses to one or more treatment variables. For instance and without limitation, treatment variable table 224 may include a user response generated in regard to the cost of a treatment.

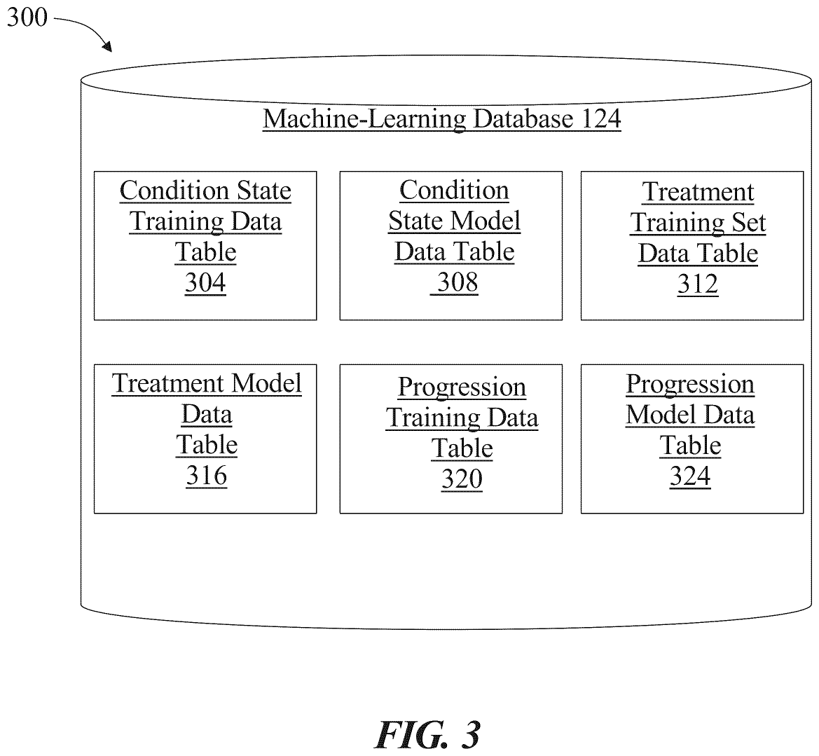

Referring now to FIG. 3, an exemplary embodiment of machine-learning database 124 is illustrated. Machine-learning database 124 may be implemented as any data structure suitable for use as user database 120 as described above in more detail. One or more tables contained within machine-learning database 124 may include condition state training data table 304; condition state training data table 304 may include one or more condition state training data 112 sets. One or more tables contained within machine-learning database 124 may include condition state model data table 308; condition state model data table 308 may include one or more condition state model 128. One or more tables contained within machine-learning database 124 may include treatment training set data table 312; treatment training set data table 312 may include one or more treatment training set 152. One or more tables contained within machine-learning database 124 may include treatment model data table 316; treatment model data table 316 may include one or more treatment model 156. One or more tables contained within machine-learning database 124 may include progression training data table 320; progression training data table 320 may include one or more progression training data 144 sets. One or more tables contained within machine-learning database 124 may include progression model data table 324; progression model data table 324 may include one or more progression models 148. In an embodiment, training sets and/or machine-learning models contained within machine-learning database 124 may be organized according to one or more categories such as for example, by condition, by treatment, by severity of condition, by progression of condition and the like. One or more machine-learning models contained within machine-learning database 124 may have been previously calculated.

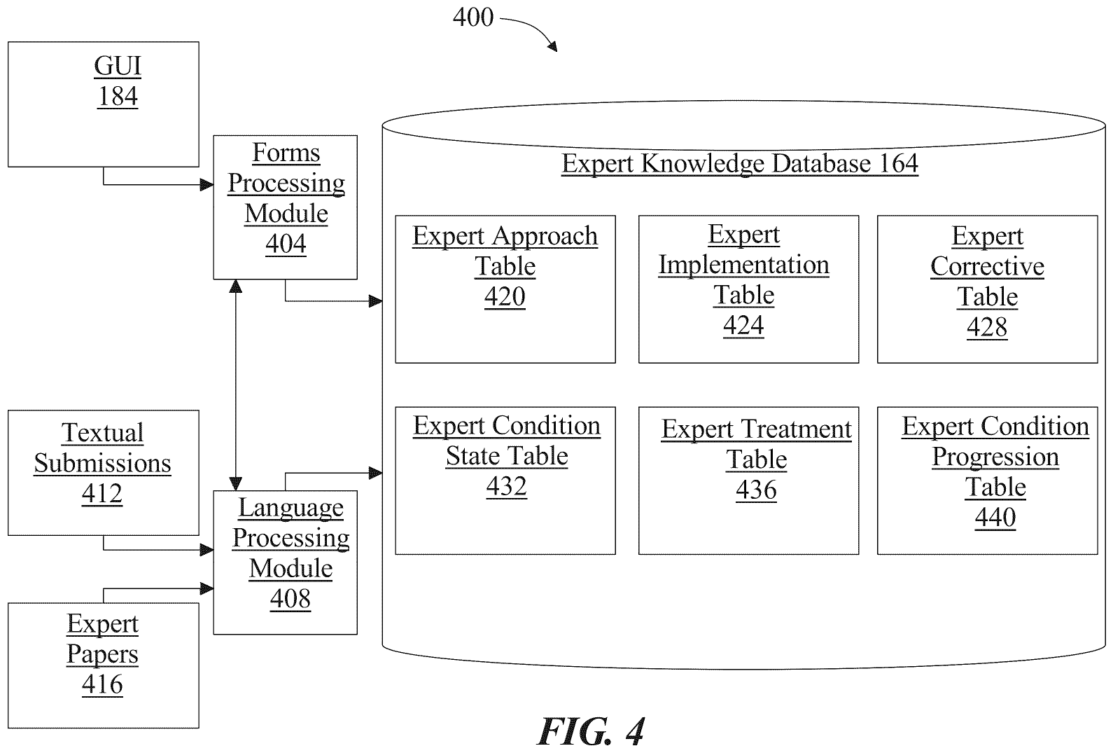

Referring now to FIG. 4, an exemplary embodiment 400 of expert knowledge database 164 is illustrated. Expert knowledge database 164 may be implemented as any data structure suitable for use as user database 120 as described above in reference to FIG. 1. One or more database tables may be linked to one another by, for instance, common column values. For instance, a common column between two tables of expert knowledge database 164 may include an identifier of an expert submission, such as a form entry, textual submission, expert paper, or the like, for instance as defined below; as a result, a query may be able to retrieve all rows from any table pertaining to a given submission or set thereof. Other columns may include any other category usable for organization or subdivision of expert data, including types of expert data, names and/or identifiers of experts submitting the data, times of submission, or the like; persons skilled in the art, upon reviewing the entirety of this disclosure, will be aware of various ways in which expert data may be included in one or more tables.

With continued reference to FIG. 4, expert knowledge database 164 includes a forms processing module 404 that may sort data entered in a submission via graphical user interface 184 by, for instance, sorting data from entries in the graphical user interface 184 to related categories of data; for instance, data entered in an entry relating in the graphical user interface 184 to a treatment may be sorted into variables and/or data structures for storage of treatments, while data entered in an entry relating to a category of condition state label 116 and/or an element thereof may be sorted into variables and/or data structures for the storage of, respectively, categories of condition state label 116. Where data is chosen by an expert from pre-selected entries such as drop-down lists, data may be stored directly; where data is entered in textual form, language processing module 408 may be used to map data to an appropriate existing label, for instance using a vector similarity test or other synonym-sensitive language processing test to map physiological data to an existing label. Alternatively or additionally, when a language processing algorithm, such as vector similarity comparison, indicates that an entry is not a synonym of an existing label, language processing module 408 may indicate that entry should be treated as relating to a new label; this may be determined by, e.g., comparison to a threshold number of cosine similarity and/or other geometric measures of vector similarity of the entered text to a nearest existent label, and determination that a degree of similarity falls below the threshold number and/or a degree of dissimilarity falls above the threshold number. Data from expert textual submissions 412, such as accomplished by filling out a paper or PDF form and/or submitting narrative information, may likewise be processed using language processing module 408. Data may be extracted from expert papers 416, which may include without limitation publications in medical and/or scientific journals, by language processing module 408 via any suitable process as described herein. Persons skilled in the art, upon reviewing the entirety of this disclosure, will be aware of various additional methods whereby novel terms may be separated from already-classified terms and/or synonyms therefore, as consistent with this disclosure.

With continued reference to FIG. 4, one or more tables contained within expert knowledge database 164 may include expert approach table 420; expert approach table 420 may include one or more data entries containing expert input regarding approach factor 168. One or more tables contained within expert knowledge database 164 may include expert implementation table 424; expert implementation table 424 may include one or more data entries containing expert input regarding implementation factor 172. One or more tables contained within expert knowledge database 164 may include expert corrective table 428; expert corrective table 428 may include one or more data entries containing expert input regarding corrective factor 176. One or more tables contained within expert knowledge database 164 may include expert condition state table 432; expert condition state table 432 may include one or more data entries containing condition states and physiological data. One or more tables contained within expert knowledge database 164 may include expert treatment table 436; expert treatment table 436 may include one or more data entries containing expert input regarding treatments for condition states. One or more tables contained within expert knowledge database 164 may include expert condition progression table 440; expert condition progression table 440 may include one or more data entries containing expert input regarding condition progression.

Referring now to FIG. 5, an exemplary embodiment of a machine-learning module 500 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 504 to generate an algorithm instantiated in hardware or software logic, data structures, and/or functions that will be performed by a computing device/module to produce outputs 508 given data provided as inputs 512; this is in contrast to a non-machine learning software program where the commands to be executed are determined in advance by a user and written in a programming language.

Still referring to FIG. 5, “training data,” as used herein, is data containing correlations that a machine-learning process may use to model relationships between two or more categories of data elements. For instance, and without limitation, training data 504 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 504 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 504 according to various correlations; correlations may indicate causative and/or predictive links between categories of data elements, which may be modeled as relationships such as mathematical relationships by machine-learning processes as described in further detail below. Training data 504 may be formatted and/or organized by categories of data elements, for instance by associating data elements with one or more descriptors corresponding to categories of data elements. As a non-limiting example, training data 504 may include data entered in standardized forms by persons or processes, such that entry of a given data element in a given field in a form may be mapped to one or more descriptors of categories. Elements in training data 504 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 504 may include one or more elements that are not categorized; that is, training data 504 may not be formatted or contain descriptors for some elements of data. Machine-learning algorithms and/or other processes may sort training data 504 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 504 to be made applicable for two or more distinct machine-learning algorithms as described in further detail below. Training data 504 used by machine-learning module 500 may correlate any input data as described in this disclosure to any output data as described in this disclosure. As a non-limiting illustrative example condition states, user data, biological extractions, treatments, treatment categories, and the like.

Further referring to FIG. 5, training data may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 500 may generate a classifier using a classification algorithm, defined as a processes whereby a computing device and/or any module and/or component operating thereon derives a classifier from training data 504. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to age groups, biological extraction groups, gender, and treatment preferences.

Still referring to FIG. 5, computing device 504 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 504 may then calculate a likelihood table by calculating probabilities of different data entries and classification labels. Computing device 504 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 5, computing device 504 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 5, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 5, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 5, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 5, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, santization may include utilizing algorithms for identifying duplicate entries or spell-check algorithms.

As a non-limiting example, and with further reference to FIG. 5, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 5, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 5, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 5, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 5, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{n e w} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{n e w} = \frac{X - {X}_{m e a n}}{{X}_{max} - {X}_{min}} .$$

Feature scaling may include standardization, where a difference between X and Xₘₑₐₙ is divided by a standard deviation σ of a set or subset of values:

$${X}_{n e w} = \frac{X - {X}_{m e a n}}{σ} .$$

Scaling may be performed using a median value of a a set or subset X_{median} and/or interquartile range (IQR), which represents the difference between the 25^{th} percentile value and the 50^{th} percentile value (or closest values thereto by a rounding protocol), such as:

$${X}_{n e w} = \frac{X - {X}_{median}}{IQR} .$$

Persons skilled in the art, upon reviewing the entirety of this disclosure, will be aware of various alternative or additional approaches that may be used for feature scaling.

Further referring to FIG. 5, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 5, machine-learning module 500 may be configured to perform a lazy-learning process 520 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 5, machine-learning processes as described in this disclosure may be used to generate machine-learning models 524. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 5, machine-learning algorithms may include at least a supervised machine-learning process 528. At least a supervised machine-learning process 528, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include inputs as described above as inputs, outputs as described above as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 504. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

With further reference to FIG. 5, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 5, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 5, machine learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 532 may not require a response variable; unsupervised processes 532 may be used to find interesting patterns and/or inferences between variables, to determine a degree of correlation between two or more variables, or the like.

Still referring to FIG. 5, machine-learning module 500 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 5, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 5, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 5, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 5, one or more processes or algorithms described above may be performed by at least a dedicated hardware unit 536. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 536 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 536 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 536 to perform one or more operations described herein, such as evaluation of model and/or algorithm outputs, one-time or iterative updates to parameters, coefficients, weights, and/or biases, and/or any other operations such as vector and/or matrix operations as described in this disclosure.

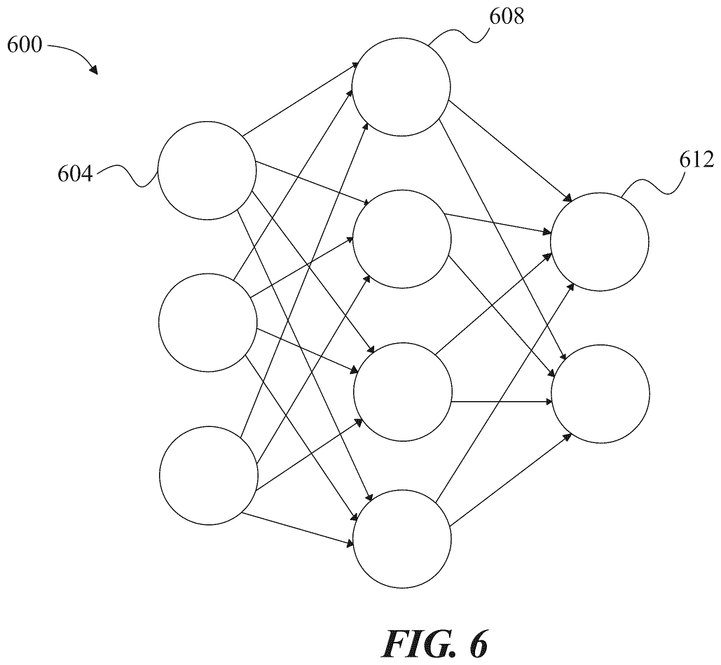

Referring now to FIG. 6, an exemplary embodiment of neural network 600 is illustrated. A neural network 600 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 604, one or more intermediate layers 608, and an output layer of nodes 612. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

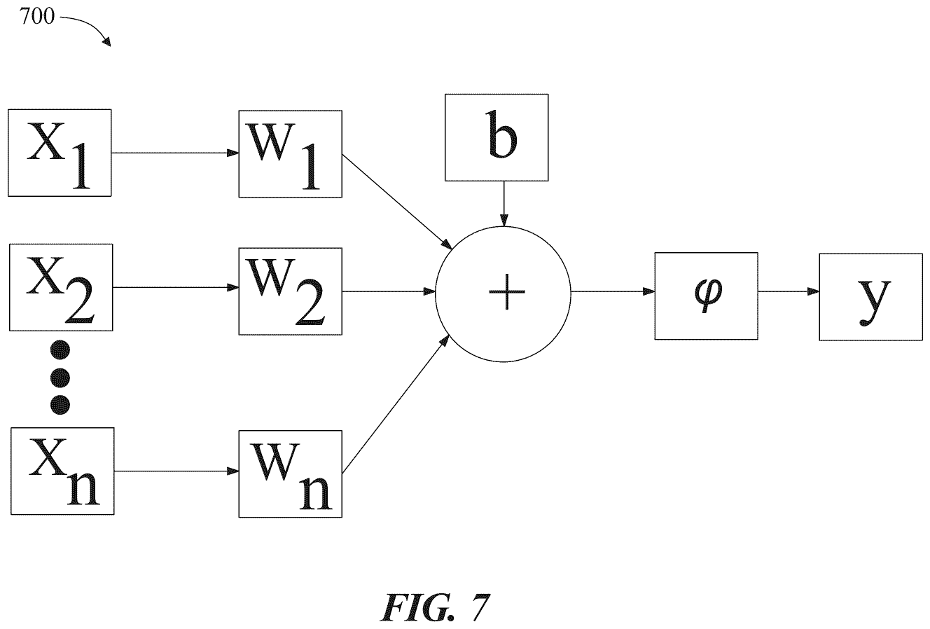

Referring now to FIG. 7, an exemplary embodiment of a node 700 of a neural network is illustrated. A node may include, without limitation a plurality of inputs x, that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as f(x)=tanh²(x), a rectified linear unit function such as f(x)=max(0, x), a “leaky” and/or “parametric” rectified linear unit function such as f(x)=max (ax, x) for some a, an exponential linear units function such as

$$f ( x ) = \{ \begin{matrix}x for x \geq 0 \\ α ( {e}^{x} - 1 ) for x < 0\end{matrix}$$

for some value of a (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

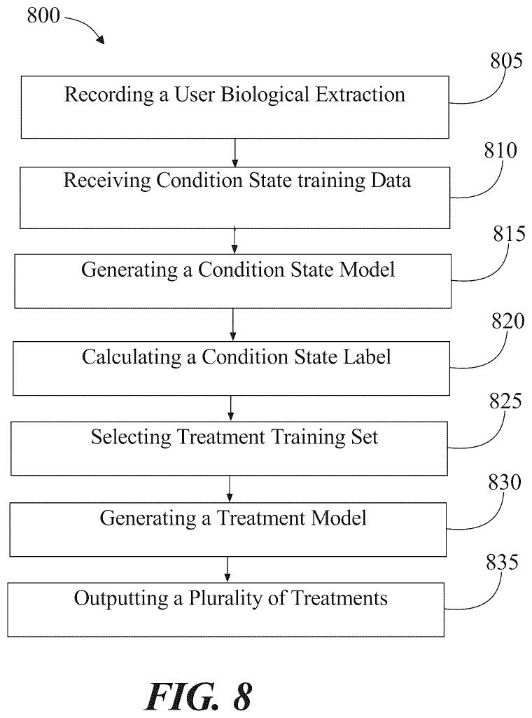

Referring now to FIG. 8, an exemplary embodiment of a method 800 of customizing treatments is illustrated. At step 805, a computing device 104 records a user biological extraction 108 containing an element of user physiological data. A user biological extraction 108 includes any of the user biological extractions as described above in reference to FIGS. 1-7. For instance and without limitation, a biological extraction 108 may include a blood sample analyzed for a user's intracellular nutrient levels. In yet another non-limiting example, a biological extraction 108 may include an element of user psychological data such as for example, a user response regarding a user's current mood and mental state. An element of user physiological data includes any of the elements of user physiological data as described above in reference to FIGS. 1-7.

With continued reference to FIG. 8, at step 810 a computing device 104 receives condition state training data 112. Condition state training data 112 includes any of the condition state training data 112 as described above in reference to FIGS. 1-7. Condition state training data 112 includes a plurality of physiological data sets and a plurality of correlated condition state label 116. In an embodiment, condition state training data 112 may be retrieved from machine-leaning database. In an embodiment, condition state training data 112 The system of claim 1, wherein receiving condition state training data 112 may be generated from a plurality of user data entries such as from previously recorded user biological extraction 108 and previously calculated condition state label 116 utilizing system 100. In such an instance, condition state training data 112 may include a plurality of user physiological data sets and a plurality of correlated user condition state label 116.

With continued reference to FIG. 8, at step 815 a computing device 104 generates a condition state model 128, using the element of user physiological data and the condition state training data 112, and using a first machine-learning algorithm. Condition state model 128 includes any of the condition state model 128 as described above in reference to FIGS. 1-7. Condition state model 128 utilizes physiological data as inputs and outputs condition state label 116. First machine-learning algorithm includes any of the machine-learning algorithms as described above. This may include for example generating one or more supervised machine-learning algorithms, one or more unsupervised machine-learning algorithms, and/or one or more lazy-learning algorithms. Selection of a particular machine-learning algorithm may be based on one or more expert inputs such as for example expert input contained within expert knowledge database 164.

With continued reference to FIG. 8, at step 820 a computing device 104 calculates a condition state label 116 for the element of user physiological data using the condition state model 128 and the element of user physiological data. Condition state label 116 includes any of the condition state label 116 as described above in reference to FIGS. 1-7. In an embodiment, a condition state label 116 describes a current, incipient, or probable future medical condition affecting a human being. For instance and without limitation, a condition state label 116 may describe a probable future medical condition such as myocardial infarction based on a user's elevated total cholesterol, elevated low density lipoprotein (LDL), dangerously low high density lipoprotein (HDL), and a positive confirmation of a medical procedure showing 90% blockage of a coronary artery. Computing device 104 may calculate a condition state label 116 to contain a current condition state progression indicator 140. Current condition state progression indicator 140 may include any of the current condition state 136 progression indicators as described above in reference to FIGS. 1-7. Current condition state progression indicator 140 may reflect how far progressed a current condition state 136 is. For instance and without limitation, a current condition state progression indicator 140 may report that a condition state such as ulcerative colitis has advanced to causing systemic complications that include sore joints and headaches. In yet another non-limiting example, a current condition state 136 progression indicator may reflect that an acute condition such as a urinary tract infection has been completely resolved following consumption of cranberry extract and d-mannose for six days. Computing device 104 may utilize a current condition state progression indicator 140 to select a treatment training set 152 and a treatment training model. In an embodiment, a treatment training set 152 and/or treatment training model may be organized within machine-learning database 124 based on progression of condition states contained within treatment training set 152 and/or treatment training models, and computing device 104 may match a current condition state progression indicator 140 to a proportionate treatment training set 152 and/or treatment training model. In an embodiment, treatment training set 152 and/or treatment training models may contain condition state progression indicator labels that indicate the progression of condition states contained within treatment training set 152 and/or treatment training models to match condition state progression indicator labels to condition state progression indicators.

With continued reference to FIG. 8, condition state progression indicator may be generated utilizing one or more machine-learning processes. Computing device 104 receives progression training data 144. Progression training data 144 includes any of the progression training data 144 as described above in reference to FIGS. 1-7. Progression training data 144 includes a plurality of physiological data sets and a plurality of correlated progression indicators. Computing device 104 generates a progression model 148 using a machine-learning algorithm, the element of user physiological data, and the progression training data 144. Progression model 148 utilizes physiological data as inputs and outputs progression indicators. Progression model 148 may be generated utilizing any of the methodologies as described above in reference to FIGS. 1-7. Computing device 104 calculates a condition state progression indicator utilizing the progression model 148.

With continued reference to FIG. 8, at step 825 computing device 104 selects a treatment training set 152 and a treatment model 156 utilizing the condition state label 116. Treatment training set 152 includes any of the treatment training set 152 as described above in reference to FIGS. 1-7. Treatment training set 152 includes a plurality of condition state label 116 and a plurality of correlated treatments. Computing device 104 may select a treatment training set 152 and/or treatment model 156 by matching an output condition state label 116 to a treatment training set 152 and/or treatment model 156 that contains the same condition state label 116. For instance and without limitation, an output condition state label 116 such as open angle glaucoma may be matched to a treatment training set 152 and/or treatment model 156 intended for open angle glaucoma. In an embodiment, treatment training set 152 and/or treatment model 156 may be stored within machine-learning database 124 according to condition state. Computing device 104 may select a treatment training set 152 and/or treatment model 156 by calculating a treatment category selector 160 and selecting a treatment training set 152 and/or treatment model 156 related to the treatment category selector 160. Treatment category selector 160 includes any of the treatment category selectors as described above in reference to FIGS. 1-7. Treatment category selector 160 includes an indication as to a particular category of treatment to be implemented and/or recommended for a particular condition identified from a condition state label 116. For instance and without limitation, a treatment category selector 160 may indicate that a condition such as metabolic syndrome is to be treated with behavior modifications and nutritional treatments. In yet another non-limiting example, a treatment category selector 160 may indicate that a condition such as pulmonary hypertension is to be treated with medication treatments and fitness treatments. Computing device 104 may select a treatment training set 152 and a treatment model 156 related to a treatment category selector 160. For instance and without limitation, computing device 104 may select a treatment training set 152 that contains treatment outputs that are of the same category of treatment contained within a treatment category selector 160. For example, computing device 104 may utilize a treatment category selector 160 that contains nutritional treatments and homeopathic treatments to select a treatment training set 152 and a treatment model 156 that contains nutritional treatments and/or homeopathic treatments. Computing device 104 may calculate treatment category selector 160 by multiplying an approach factor 168 multiplied by an implementation factor 172 multiplied by a corrective factor 176. Factors utilized to calculate treatment category selector 160 may contain numerical values that may be utilized to select an output containing a treatment training set 152 and/or treatment model 156. This may be performed as described above in more detail in reference to FIG. 1. Computing device 104 may select a treatment training set 152 and/or a treatment model 156 based on a current treatment input descriptor 180 received from a remote device 132. Current treatment input descriptor 180 includes any of the current treatment input descriptor 180 as described above in reference to FIGS. 1-7. In an embodiment, current treatment input descriptor 180 may include a description of one or more current treatments the user may be currently practicing and/or implementing. Current treatment input descriptor 180 may be received by computing device 104 from a remote device 132 operated by a user utilizing any network methodology and transmission as described herein. Computing device 104 may locate a treatment training set 152 and/or treatment model 156 as a function of a current treatment input descriptor 180. For example, computing device 104 may locate a treatment model 156 that contains output treatments that further expand and/or build upon treatments a user may be currently engaging in. For example, computing device may select a treatment model 156 that contains additional exercises that increase flexibility for a user who is currently practicing yoga three times each week. In an embodiment, computing device 104 may utilize a current treatment input descriptor 180 to select additional categories of treatment that a user may implement. For example, computing device 104 may select a treatment model 156 that contains additional treatment categories such as dietary treatments and behavior modifications for a user who is currently engaged in prescription therapies.

With continued reference to FIG. 8, at step 830, computing device 104 generates a treatment model 156 utilizing a machine-learning algorithm, a condition state label 116 and selected treatment training data. Computing device 104 may generate a treatment model 156 utilizing any of the methodologies as described above in reference to FIGS. 1-7. Generating treatment model 156 may include calculating one or more machine-learning algorithms. Machine-learning algorithms include any of the machine-learning algorithms as described above in reference to FIGS. 1-7. Treatment model 156 utilizes a condition state label 116 as an input and outputs a plurality of treatments. Treatments include any of the treatments as described above in reference to FIGS. 1-7.

With continued reference to FIG. 8, at step 835, computing device 104 outputs a plurality of treatments utilizing the treatment model 156. Treatments include any of the treatments as described above in reference to FIGS. 1-7. Computing device 104 may select a treatment from the plurality of output treatments by generating a loss function. Computing device 104 may receive user variables from a remote device 132 operated by a user relating to the plurality of output treatments. Variables may include one or more user responses in regard to different aspects of treatment. For example, variables may include one or more user views regarding cost which may indicate how much money a user is willing to spend on a particular treatment. Variables may include other user inputs regarding treatments such as how much time each day a user is willing to devote to a treatment, how adherent a user is with previous treatments, how far a user is willing to travel to receive treatment, how intense of treatment a user seeks to engage in, a user's views on functional medicine treatments versus conventional treatments and the like. Computing device 104 may utilize variables to generate a loss function utilizing the user variables and minimize the loss function. This may be performed utilizing any of the methodologies as described above in reference to FIGS. 1-7. Computing device 104 selects a treatment from a plurality of output treatments as a function of minimizing the loss function.

With continued reference to FIG. 1, computing device 104 may utilize generated inputs and outputs in machine-learning models and/or machine-learning algorithms to update subsequent uses of system 100. Computing device 104 incorporates output treatments and a user biological extraction 108 into a treatment training set 152. In an embodiment, an updated treatment training set 152 that contains output treatments and a user biological extraction 108 may be stored in machine-learning database 124 to be utilized in subsequent machine-learning algorithms and models. Computing device 104 may incorporate a condition state label 116 generated for an element of user physiological data into condition state training data 112. In an embodiment, computing device 104 may incorporate an element of user physiological data and a generated conditions state label into condition state training data 112. Updated training sets may be sorted within machine-learning database 124.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

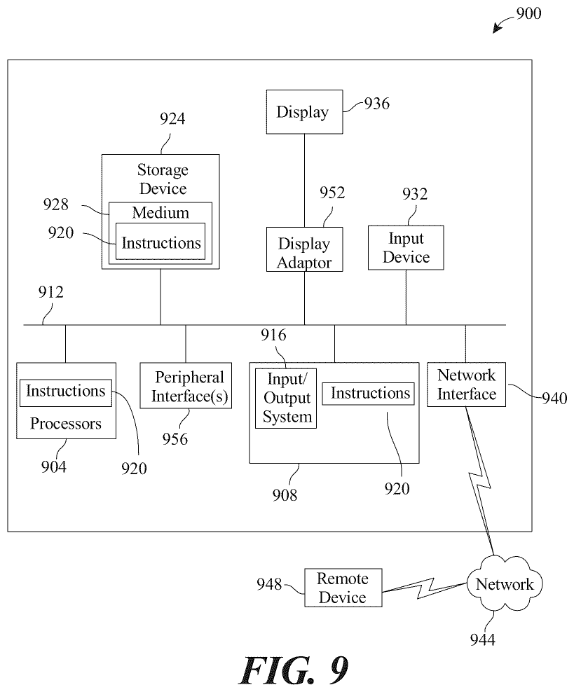

FIG. 9 shows a diagrammatic representation of one embodiment of a computing device in the exemplary form of a computer system 900 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 900 includes a processor 904 and a memory 908 that communicate with each other, and with other components, via a bus 912. Bus 912 may include any of several types of bus structures including, but not limited to, a memory bus, a memory controller, a peripheral bus, a local bus, and any combinations thereof, using any of a variety of bus architectures.

Processor 904 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 904 may be organized according to Von Neumann and/or Harvard architecture as a non-limiting example. Processor 904 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 908 may include various components (e.g., machine-readable media) including, but not limited to, a random-access memory component, a read only component, and any combinations thereof. In one example, a basic input/output system 916 (BIOS), including basic routines that help to transfer information between elements within computer system 900, such as during start-up, may be stored in memory 908. Memory 908 may also include (e.g., stored on one or more machine-readable media) instructions (e.g., software) 920 embodying any one or more of the aspects and/or methodologies of the present disclosure. In another example, memory 908 may further include any number of program modules including, but not limited to, an operating system, one or more application programs, other program modules, program data, and any combinations thereof.

Computer system 900 may also include a storage device 924. Examples of a storage device (e.g., storage device 924) include, but are not limited to, a hard disk drive, a magnetic disk drive, an optical disc drive in combination with an optical medium, a solid-state memory device, and any combinations thereof. Storage device 924 may be connected to bus 912 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 924 (or one or more components thereof) may be removably interfaced with computer system 900 (e.g., via an external port connector (not shown)). Particularly, storage device 924 and an associated machine-readable medium 928 may provide nonvolatile and/or volatile storage of machine-readable instructions, data structures, program modules, and/or other data for computer system 900. In one example, software 920 may reside, completely or partially, within machine-readable medium 928. In another example, software 920 may reside, completely or partially, within processor 904.

Computer system 900 may also include an input device 932. In one example, a user of computer system 900 may enter commands and/or other information into computer system 900 via input device 932. Examples of an input device 932 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 932 may be interfaced to bus 912 via any of a variety of interfaces (not shown) including, but not limited to, a serial interface, a parallel interface, a game port, a USB interface, a FIREWIRE interface, a direct interface to bus 912, and any combinations thereof. Input device 932 may include a touch screen interface that may be a part of or separate from display 936, discussed further below. Input device 932 may be utilized as a user selection device for selecting one or more graphical representations in a graphical interface as described above.

A user may also input commands and/or other information to computer system 900 via storage device 924 (e.g., a removable disk drive, a flash drive, etc.) and/or network interface device 940. A network interface device, such as network interface device 940, may be utilized for connecting computer system 900 to one or more of a variety of networks, such as network 944, and one or more remote devices 948 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 944, may employ a wired and/or a wireless mode of communication. In general, any network topology may be used. Information (e.g., data, software 920, etc.) may be communicated to and/or from computer system 900 via network interface device 940.

Computer system 900 may further include a video display adapter 952 for communicating a displayable image to a display device, such as display device 936. Examples of a display device include, but are not limited to, a liquid crystal display (LCD), a cathode ray tube (CRT), a plasma display, a light emitting diode (LED) display, and any combinations thereof. Display adapter 952 and display device 936 may be utilized in combination with processor 904 to provide graphical representations of aspects of the present disclosure. In addition to a display device, computer system 900 may include one or more other peripheral output devices including, but not limited to, an audio speaker, a printer, and any combinations thereof. Such peripheral output devices may be connected to bus 912 via a peripheral interface 956. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for customizing treatments, the system comprising:
a computing device, the computing device designed and configured to:
calculate a condition state label as a function of an element of user physiological data;
generate a treatment model, using a second machine-learning algorithm and a treatment training set, wherein the treatment model utilizes condition state labels as inputs and outputs treatments, wherein generating the treatment model further comprises:
calculating a treatment category selector as a function of an implementation factor, wherein the implementation factor indicates a user preference pertaining to different treatment practices;
output a treatment utilizing the treatment model to a remote device;
receive a treatment response from a remote device; and
generate a treatment response score as a function of the treatment response.

2. The system of claim 1, wherein the computing device is further configured to calculate a current condition state progression indicator, wherein calculating the current condition state progression indicator comprises generating a progression model, wherein the progression model comprises a second machine-learning model trained by progression training data comprising a plurality of physiological data sets and a plurality of correlated progression indicators and wherein the progression model is configured to receive the element of user physiological data as an input and output a current condition state progression indicator.

3. The system of claim 1, wherein:
the computing device is further configured to categorize the treatment response to a treatment response category; and
generating the treatment response score as a function of the treatment response comprises generating the treatment response score as a function of treatment response and the treatment response category.

4. The system of claim 3, wherein categorizing the treatment response to the treatment response category comprises:
receiving treatment response training data, wherein the treatment response training data comprises a plurality of treatment responses correlated to treatment response categories;
training a treatment response classifier using the treatment response training data; and
categorizing the treatment response to the treatment response category using the trained treatment response classifier.

5. The system of claim 1, wherein the computing device is further configured to generate an encouragement notification as a function of comparing the treatment response score to a treatment response score threshold.

6. The system of claim 5, wherein generating the encouragement notification as a function of comparing the treatment response score to the treatment response score threshold comprises generating the encouragement notification using a large language model (LLM).

7. The system of claim 1, wherein the computing device is further configured to generate a healthcare notification, wherein generating the healthcare notification comprises:
selecting the healthcare professional as a function of the treatment category selector; and
inserting contact information associated with the healthcare professional into the healthcare notification.

8. The system of claim 1, wherein receiving the treatment response from the remote device comprises:
generating a treatment response form and transmitting the treatment response form to the remote device on a set periodic basis;
receiving a completed treatment response form from the remote device; and
extracting the treatment response from the completed treatment response form.

9. The system of claim 1, wherein calculating the treatment category selector further comprises multiplying an approach factor by the implementation factor and a corrective factor.

10. The system of claim 1, wherein generating the treatment response score as a function of the treatment response comprises:
receiving score training data, wherein the score training data comprises treatment responses correlated to treatment response scores;
training a score machine-learning model using the score training data; and
generating the treatment response score using the trained score machine-learning model.

11. A method for customizing treatments, the method comprising:
calculating, by the computing device, a condition state label as a function of an element of user physiological data;
generating, by the computing device, a treatment model, using a second machine-learning algorithm and a treatment training set, wherein the treatment model utilizes condition state labels as inputs and outputs treatments, wherein generating the treatment model further comprises:
calculating a treatment category selector as a function of an implementation factor, wherein the implementation factor indicates a user preference pertaining to different treatment practices;
outputting, by the computing device, a treatment utilizing the treatment model to a remote device;
receiving, by the computing device, a treatment response from a remote device; and
generating, by the computing device, a treatment response score as a function of the treatment response.

12. The method of claim 1, further comprising calculating a current condition state progression indicator, wherein calculating the current condition state progression indicator comprises generating a progression model, wherein the progression model comprises a second machine-learning model trained by progression training data comprising a plurality of physiological data sets and a plurality of correlated progression indicators and wherein the progression model is configured to receive the element of user physiological data as an input and output a current condition state progression indicator.

13. The method of claim 11, wherein:
the method further comprises categorizing, by the computing device, the treatment response to a treatment response category; and
generating the treatment response score as a function of the treatment response comprises generating the treatment response score as a function of treatment response and the treatment response category.

14. The method of claim 13, wherein categorizing the treatment response to the treatment response category comprises:
receiving treatment response training data, wherein the treatment response training data comprises a plurality of treatment responses correlated to treatment response categories;
training a treatment response classifier using the treatment response training data; and
categorizing the treatment response to the treatment response category using the trained treatment response classifier.

15. The method of claim 11, further comprising generating, by the computing device, an encouragement notification as a function of comparing the treatment response score to a treatment response score threshold.

16. The method of claim 15, wherein generating the encouragement notification as a function of comparing the treatment response score to the treatment response score threshold comprises generating the encouragement notification using a large language model (LLM).

17. The method of claim 11, further comprising generating a healthcare notification, wherein generating the healthcare notification comprises:
selecting the healthcare professional as a function of the treatment category selector; and
inserting contact information associated with the healthcare professional into the healthcare notification.

18. The method of claim 11, wherein receiving the treatment response from the remote device comprises:
generating a treatment response form and transmitting the treatment response form to the remote device on a set periodic basis;
receiving a completed treatment response form from the remote device; and
extracting the treatment response from the completed treatment response form.

19. The method of claim 11, wherein calculating the treatment category selector further comprises multiplying an approach factor by the implementation factor and a corrective factor.

20. The method of claim 11, wherein generating the treatment response score as a function of the treatment response comprises:
receiving score training data, wherein the score training data comprises treatment responses correlated to treatment response scores;
training a score machine-learning model using the score training data; and
generating the treatment response score using the trained score machine-learning model.
